(19) United States
(12) Patent Application Publication
(10) Pub. No.: US 2026/0183391 A1
(43) Pub. Date: Jul. 2, 2026

(54) Methods of Treating Cervical Cancer Using Anti-HER2 Antibody-Drug Conjugates

(71) Applicant: RemeGen Co., Ltd., Yantai (CN)

(72) Inventors: Jianmin Fang, Yantai (CN); Xiaohong Su, Yantai (CN); Yan Wang, Yantai (CN)

(73) Assignee: RemeGen Co., Ltd., Yantai (CN)

(21) Appl. No.: 19/564,445

(22) Filed: Mar. 12, 2026

Related U.S. Application Data
(63) Continuation of application No. PCT/CN2024/109209, filed on Aug. 1, 2024.

(30) Foreign Application Priority Data
Sep. 14, 2023 (WO) PCT/CN23/118894

(51) Int. Cl. A61K 39/395; A61K 39/00; A61K 47/68; A61P 35/00

(52) U.S. Cl. CPC A61K 39/3955; A61K 47/68031; A61K 47/6849; A61P 35/00; A61K 2039/507; A61K 2039/545

(57) ABSTRACT

Provided herein are methods for treating or preventing progression of cervical cancer in an individual, including administering to the individual an effective amount of an anti-human epidermal growth factor receptor 2 (HER2) antibody-drug conjugate that comprises an anti-HER2 antibody and a cytotoxic molecule. Also provided herein are methods for treating or preventing progression of cervical cancer in an individual, including administering to the individual an effective amount of an anti-HER2 antibody-drug conjugate that includes an anti-HER2 antibody and a cytotoxic molecule, in combination with an anti-PD-1 antibody. Also provided herein are uses of an anti-HER2 antibody-drug conjugate, or an anti-HER2 antibody drug conjugate in combination with an anti-PD-1 antibody, for manufacture of a medicament for treating or preventing progression of a previously treated cervical cancer in an individual. Also provided herein are pharmaceutical compositions for use in treating a previously treated cervical cancer in an individual.

### CROSS-REFERENCE TO RELATED APPLICATIONS

This application is a continuation application of International Application No. PCT/CN2024/109209, filed Aug. 1, 2024, which claims the benefit of priority of International Application No. PCT/CN2023/118894, filed Sep. 14, 2023, the contents of each of which are hereby incorporated by reference in their entirety.

### REFERENCE TO AN ELECTRONIC SEQUENCE LISTING

This application contains an electronic sequence listing, which has been submitted electronically in .xml format. The contents of the electronic sequence listing (file name: 761682010641 seqlist.xml; size: 22,236 bytes; and date of creation: Jul. 29, 2024) are herein incorporated by reference in their entirety.

### FIELD

The present disclosure relates to methods for treating or preventing progression of cervical cancer in an individual, as well as compositions, uses, and kits related thereto. In some embodiments, the methods comprise administering to the individual an effective amount of an anti-human epidermal growth factor receptor 2 (HER2) antibody-drug conjugate that comprises an anti-HER2 antibody and a cytotoxic molecule.

### BACKGROUND

ErbB2, also known as HER2/neu, is a member of a family of tyrosine kinases that regulate cell growth and survival (Lengyel, C. G. et al. (2021) Gastrointest Disord 3 (1): 1-22). Overexpression and/or amplification of HER2 is seen in many malignancies including cervical, breast, gastric, ovarian, pancreatic, colorectal, and endometrial cancer (Neve, R. M. et al. (2001) Ann Oncol 12 (Suppl1): S9-S13; Menard, S. et al. (2003) Oncogene 22 (42): 6570-6578; Moasser, M. M. (2007) Oncogene 26 (45): 6469-6487; Iqbal, N. et al. (2014) Mol Biol Int 2014:852748). Tumors that overexpress HER2 are thought to be more aggressive and associated with poorer overall survival (OS) compared to HER2-negative cancers. Cancers characterized by overexpression of HER2 (e.g., HER2-positive cancers) are often correlated with poor prognosis and/or are resistant to many standard therapies. As such, a handful of therapies targeting HER2 have been approved for treatment of HER2-positive cancer, including antibodies such as trastuzumab and pertuzumab, antibody-drug conjugates (ADCs) such as trastuzumab maytansine (T-DM1), and small molecule tyrosine kinase inhibitors (TKIs) such as lapatinib, pazopanib, afatinib, and neratinib.

The introduction of HER2-targeted therapy using either antibody-based therapies or small molecule tyrosine kinase inhibitors (TKI) has led to significant and ongoing improvements in disease-free survival (DFS), progression-free survival (PFS), and OS in both the neoadjuvant/adjuvant and metastatic settings (Slamon, D. J. et al. (2001) N Engl J Med 344 (11): 783-792; Geyer, C. E. et al. (2006) N Engl J Med 355 (26): 2733-2743; Baselga 2012; Verma 2012). HER2 is an effective therapeutic target in multiple solid tumors with anti-HER2 biologic agents, antibody-drug conjugates (ADC), and small molecule drugs approved for patients with HER2 overexpressing/amplified (hereafter HER2+) breast and gastric cancers. HER2 expression occurs commonly in many tumors.

With the development of ADC, HER2 expression can be utilized as target to direct the cytotoxic drug to the tumor cells. After binding of an HER2 directed ADC to HER2 on the cell surface, the ADC-receptor complex internalizes and traffics through the endo-lysosomal pathway. The subsequent release of cytotoxic payload eventually leads to cell apoptosis. New generations of ADCs with cleavable linkers may exhibit a bystander effect and have shown encouraging single agent activity in multiple HER2 expressing advanced tumors including breast cancer, gastric cancer, urothelial cancer. The data on HER2 directed ADCs observed to date suggest the potential for clinically meaningful efficacy in HER2 expressing tumors, and safety and efficacy data demonstrated a favorable benefit-risk profile which supports further development of HER2 directed ADCs in a broader range of tumor types with HER2 expression.

Cervical cancer poses a significant medical problem worldwide with an estimated incidence of more than 600,000 new cases and 325,000 deaths annually. See Sung et al., 2020, CA Cancer J Clin., 71:209-249; Arbyn et al., 2020 Lancet Glob Health, 8: e191-e203. The main types of cervical cancer are squamous cell carcinoma and adenocarcinoma. Long-lasting infections with human papillomavirus (HPV) type 16 and 18 cause most cases of cervical cancer. In metastatic/recurrent cervical cancer, the 5-year overall survival (OS) rate is only 17%, and the estimated OS is around 13-17 months. See Cibula et al., 2018, Int J Gynecol Cancer, 28: 641-655; Gennigens et al., 2022, ESMO Open. 7 (5): 100579. As such, there remains a need for novel therapies for metastatic/recurrent cervical cancer that can provide clinical benefit.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the cervical cancer is selected from the group consisting of squamous cell carcinoma and adenocarcinoma.

In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the cervical cancer is selected from the group consisting of squamous cell carcinoma and adenocarcinoma.

In some embodiments according to any of the embodiments described herein, the cervical cancer is squamous cell carcinoma. In some embodiments, according to any of the embodiments described herein, the cervical cancer is adenocarcinoma.

In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein administration of the antibody-drug conjugate results in a complete response (CR) or partial response (PR) in the individual.

In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein administration of the antibody-drug conjugate results in a complete response (CR) or partial response (PR) in the individual . . .

In some embodiments according to any of the embodiments described herein, the VH domain comprises the amino acid sequence of SEQ ID NO: 7, and the VL domain comprises the amino acid sequence of SEQ ID NO: 8. In some embodiments according to any of the embodiments described herein, the VH domain comprises the amino acid sequence of SEQ ID NO: 7. In some embodiments, the VL domain comprises the amino acid sequence of SEQ ID NO: 8. In some embodiments, the heavy chain comprises the amino acid sequence of SEQ ID NO: 9 or SEQ ID NO: 10, and the light chain comprises the amino acid sequence of SEQ ID NO: 11. In some embodiments, the heavy chain comprises the amino acid sequence of SEQ ID NO: 9. In some embodiments, the heavy chain comprises the amino acid sequence of SEQ ID NO: 10. In some embodiments, the light chain comprises the amino acid sequence of SEQ ID NO: 11.

In some embodiments according to any of the embodiments described herein, the cytotoxic molecule comprises a tubulin inhibitor or DNA damaging agent. In some embodiments, the cytotoxic molecule comprises a tubulin inhibitor, wherein the tubulin inhibitor comprises a dolastatin or derivative thereof, an auristatin or derivative thereof, or a maytansinoid or derivative thereof. In some embodiments, the tubulin inhibitor comprises monomethyl auristatin E (MMAE), monomethyl auristatin F (MMAF), or auristatin F (AF). In some embodiments, the tubulin inhibitor comprises emtansine (DM1), maytansine (DM3), or ravtansine (DM4). In some embodiments, the cytotoxic molecule comprises a DNA damaging agent, wherein the DNA damaging agent comprises a calicheamicin, duocarmycin, pyrrolobenzodiazepine (PBD), or SN-38. In some embodiments, the cytotoxic molecule comprises an amanitin, anthracycline, baccatin, camptothecin, cemadotin, colchicine, colcemid, combretastatin, cryptophycin, discodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid. In some embodiments, the antibody-drug conjugate is represented by formula Ab-(L-U) n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. In some embodiments, the linker is attached to the anti-HER2 antibody via a thiol or amino moiety. In some embodiments, the linker is selected from the group consisting of maleimidocaproyl valine citrulline p-amino-benzyl (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl) propanamido) ethoxy) ethoxy) propanoate), Mal-di-EG-OSu (2,5-dioxopyrrolidin-1-yl 3-(2-(2-(2,5-dioxo-2,5-dihydro-1H-pyrrol-1-yl) ethoxy) ethoxy) propanoate), Mal-Tri-EG-OSu (2,5-dioxopyrrolidin-1-yl 3-(2-(2-(2-(2,5-dioxo-2,5-dihydro-1H-pyrrol-1-yl) ethoxy) ethoxy) ethoxy) ethoxypropanoate), Mal-Tetra-EG-OSu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-OSu (2,5-dioxopyrrolidin-1-yl 3 (2-(2-(2-bromoacetamido) ethoxy) ethoxy) propanoate), Py-ds-prp-OSu (2-5-dioxopyrrolidin-1-yl 3-(pyridine-2-yldisulfanyl) propanoate), Py-ds-Prp-OPEP (perfluorophenyl 3-(pyridine-2-yldisulfanyl) propanoate), Py-ds-dmBut-OSu (2,5-dioxopyrrolidin-1-yl 4-methyl-4-(pyridine-2-yldisulfanyl) pentanoate, Py-ds-dmBut-OPF (perfluorophenyl 4-methyl-4-(pyridine-2-yldisulfanyl) pentanoate), SMCC (N-succinimidyl 4-(maleimidomethyl)cyclohexanecarboxylate), MBS (3-maleimidobenzoic acid N-hydroxysuccinimide ester), SATA (S—(N-succinimidyl) thioacetate), SPDP ((N-succinimidyl 3-(2-pyridyldithio) propionate), and SMPT ((N-succinimidyloxy carbonyl)-1-methyl-1-(2-pyridyldithio) toluene). In some embodiments, the antibody-drug conjugate is disitamab vedotin.

In some embodiments according to any of the embodiments described herein, the cancer is recurrent or metastatic cervical cancer. In some embodiments, the cancer has been previously treated. In some embodiments, the cancer has been previously treated with at least first-line platinum-based standard therapy. In some embodiments, the cancer has been previously treated with radiotherapy. In some embodiments, administration of the antibody-drug conjugate is second-line therapy, third-line therapy, or fourth-line therapy. In some embodiments, cells of the cancer express HER2. In some embodiments, the cancer is a HER2-positive cancer. In some embodiments, a sample obtained from the individual comprises cancer cells that express HER2 on their cell surface at a level of IHC≥2+, as measured by IHC assay, and are positive for HER2 expression by fluorescence in situ hybridization (FISH+). In some embodiments, a sample obtained from the individual comprises cancer cells that express HER2 on their cell surface at a level of IHC>3+, as measured by IHC assay. In some embodiments, the cancer is HER2 low. In some embodiments, a sample obtained from the individual comprises cancer cells that express HER2 on their cell surface at a level of IHC2+, as measured by IHC assay, and is negative for HER2 expression by fluorescence in situ hybridization (FISH−). In some embodiments, a sample obtained from the individual comprises cancer cells that express HER2 on their cell surface at a level of IHC1+, as measured by IHC assay. In some embodiments, the cancer has a primary FIGO stage of IIB or above.

In some embodiments according to any of the embodiments described herein, the antibody-drug conjugate is administered to the individual at a dose of 2.0 mg/kg. In some embodiments, the dose is measured using the Bovine serum albumin (BSA)-based Extinction Coefficient (EC) method. In some embodiments according to any of the embodiments described herein, the antibody-drug conjugate is administered to the individual at a dose of 2.0 mg/kg, wherein the dose is measured using the Bovine serum albumin (BSA)-based Extinction Coefficient (EC) method. In some embodiments, the antibody-drug conjugate is administered intravenously to the individual. In some embodiments, the antibody-drug conjugate is administered to the individuals every 2 weeks or every 14 days.

In some embodiments according to any of the embodiments described herein, the cervical cancer is selected from the group consisting of squamous cell carcinoma and adenocarcinoma. In some embodiments, the cervical cancer is squamous cell carcinoma. In some embodiments, the cervical cancer is adenocarcinoma.

In some embodiments according to any of the embodiments described herein, administration of the antibody-drug conjugate results in a complete response (CR) or partial response (PR) in the individual.

In some embodiments according to any of the embodiments described herein, the time to onset of effect is less than two months. In some embodiments, the time to onset of effect is less than or equal to 1.5 months. In some embodiments, the duration of response is longer than five months. In some embodiments, the duration of progression free survival is longer than four months.

In some embodiments according to any of the embodiments described herein, the antibody-drug conjugate is disitamab vedotin, and the method comprises intravenously administering disitamab vedotin to the individual at 2 mg/kg every two weeks or 14 days. In some embodiments according to any of the embodiments described herein, the individual is a human.

In some aspects, provided herein are uses of an anti-HER2 antibody-drug conjugate for manufacture of a medicament for treating or preventing progression of a previously treated cancer in an individual, wherein the use comprises administering an effective amount of the anti-HER2 antibody-drug conjugate according to the method of any one of the embodiments described herein.

In some aspects, provided herein are pharmaceutical compositions for use in treating a previously treated cancer in an individual, wherein the use comprises administering an effective amount of the anti-HER2 antibody-drug conjugate according to the method of any one of the embodiments described herein.

In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule, and an effective amount of an anti-PD-1 antibody; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the anti-PD-1 antibody comprises a heavy chain comprising a heavy chain variable (VH) domain and a light chain comprising a light chain variable (VL) domain, wherein the VH domain comprises a CDR-H1 comprising the amino acid sequence STTYYWV (SEQ ID NO: 17), a CDR-H2 comprising the amino acid sequence SISYSGSTYYNPSLKS (SEQ ID NO: 18), and a CDR-H3 comprising the amino acid sequence HLGYNGRYLPFDY (SEQ ID NO: 19) and the VL domain comprises a CDR-L1 comprising the amino acid sequence TGTSSDVGFYNYVS (SEQ ID NO: 20), a CDR-L2 comprising the amino acid sequence DVSNRPS (SEQ ID NO: 21), and a CDR-L3 comprising the amino acid sequence SSYTSISTWV (SEQ ID NO: 22). In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule, and an effective amount of an anti-PD-1 antibody; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the anti-PD-1 antibody comprises a heavy chain comprising a heavy chain variable (VH) domain and a light chain comprising a light chain variable (VL) domain, wherein the VH domain comprises a CDR-H1 comprising the amino acid sequence STTYYWV (SEQ ID NO: 17), a CDR-H2 comprising the amino acid sequence SISYSGSTYYNPSLKS (SEQ ID NO: 18), and a CDR-H3 comprising the amino acid sequence HLGYNGRYLPFDY (SEQ ID NO: 19) and the VL domain comprises a CDR-L1 comprising the amino acid sequence TGTSSDVGFYNYVS (SEQ ID NO: 20), a CDR-L2 comprising the amino acid sequence DVSNRPS (SEQ ID NO: 21), and a CDR-L3 comprising the amino acid sequence SSYTSISTWV (SEQ ID NO: 22).

In some embodiments of the above aspects, the VH domain of the anti-HER2 antibody comprises the amino acid sequence of SEQ ID NO: 7 and the VL domain of the anti-HER2 antibody comprises the amino acid sequence of SEQ ID NO: 8. In some embodiments of the above aspects, the VH domain of the anti-HER2 antibody comprises the amino acid sequence of SEQ ID NO: 7. In some embodiments of the above aspects, which may be combined with any of the previous embodiments, the VL domain of the anti-HER2 antibody comprises the amino acid sequence of SEQ ID NO: 8. In some embodiments of the above aspects, which may be combined with any of the previous embodiments, the heavy chain of the anti-HER2 antibody comprises the amino acid sequence of SEQ ID NO: 9 or SEQ ID NO: 10, and the light chain of the anti-HER2 antibody comprises the amino acid sequence of SEQ ID NO: 11. In some embodiments of the above aspects, which may be combined with any of the previous embodiments, the heavy chain of the anti-HER2 antibody comprises the amino acid sequence of SEQ ID NO: 9 or SEQ ID NO: 10. In some embodiments of the above aspects, which may be combined with any of the previous embodiments, the light chain of the anti-HER2 antibody comprises the amino acid sequence of SEQ ID NO: 11. In some embodiments of the above aspects, which may be combined with any of the previous embodiments, the cytotoxic molecule comprises a tubulin inhibitor or DNA damaging agent. In a further embodiment of the above aspects, the cytotoxic molecule comprises a tubulin inhibitor, wherein the tubulin inhibitor comprises a dolastatin or derivative thereof, an auristatin or derivative thereof, or a maytansinoid or derivative thereof. In another embodiment of the above aspects, the tubulin inhibitor comprises monomethyl auristatin E (MMAE), monomethyl auristatin F (MMAF), or auristatin F (AF). In an additional embodiment of the above aspects, the tubulin inhibitor comprises emtansine (DM1), maytansine (DM3), or ravtansine (DM4). In yet another embodiment, the tubulin inhibitor comprises emtansine (DM1), maytansine (DM3), or ravtansine (DM4). In some embodiments of the above aspects, which may be combined with any of the previous embodiments, the cytotoxic molecule comprises an amanitin, anthracycline, baccatin, camptothecin, cemadotin, colchicine, colcemid, combretastatin, cryptophycin, discodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid. In some embodiments of the above aspects, which may be combined with any of the previous embodiments, the antibody-drug conjugate is represented by formula Ab-(L-U) n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. In a further embodiment of the above aspects, the linker is attached to the anti-HER2 antibody via a thiol or amino moiety. In some embodiments of the above aspects, which may be combined with any of the previous embodiments, the linker is selected from the group consisting of maleimidocaproyl valine citrulline p-amino-benzyl (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl) propanamido) ethoxy) ethoxy) propanoate), Mal-di-EG-OSu (2,5-dioxopyrrolidin-1-yl 3-(2-(2-(2,5-dioxo-2,5-dihydro-1H-pyrrol-1-yl) ethoxy) ethoxy) propanoate), Mal-Tri-EG-OSu (2,5-dioxopyrrolidin-1-yl 3-(2-(2-(2-(2,5-dioxo-2,5-dihydro-1H-pyrrol-1-yl) ethoxy) ethoxy) ethoxy) ethoxypropanoate), Mal-Tetra-EG-OSu (2,5-dioxopyrrolidin-1-yl 1-(2,5-dioxo-2,5-dihydro-1H-pyrrol-1-yl)-3-oxo-7,10,13, 16-tetraoxa-4-azanonadecan-19-oate), Br-di-EG-OSu (2,5-dioxopyrrolidin-1-yl 3 (2-(2-(2-bromoacetamido) ethoxy) ethoxy) propanoate), Py-ds-prp-OSu (2-5-dioxopyrrolidin-1-yl 3-(pyridine-2-yldisulfanyl) propanoate), Py-ds-Prp-OPEP (perfluorophenyl 3-(pyridine-2-yldisulfanyl) propanoate), Py-ds-dmBut-OSu (2,5-dioxopyrrolidin-1-yl 4-methyl-4-(pyridine-2-yldisulfanyl) pentanoate, Py-ds-dmBut-OPF (perfluorophenyl 4-methyl-4-(pyridine-2-yldisulfanyl) pentanoate), SMCC (N-succinimidyl 4-(maleimidomethyl)cyclohexanecarboxylate), MBS (3-maleimidobenzoic acid N-hydroxysuccinimide ester), SATA (S—(N-succinimidyl) thioacetate), SPDP ((N-succinimidyl 3-(2-pyridyldithio) propionate), and SMPT ((N-succinimidyloxy carbonyl)-1-methyl-1-(2-pyridyldithio) toluene). In some embodiments of the above aspects, which may be combined with any of the previous embodiments, the antibody-drug conjugate is disitamab vedotin.

In some embodiments of the above aspects, which may be combined with any of the previous embodiments, the VH domain of the anti-PD-1 antibody comprises the amino acid sequence of SEQ ID NO: 14, and the VL domain of the anti-PD-1 antibody comprises the amino acid sequence of SEQ ID NO: 16. In some embodiments of the above aspects, which may be combined with any of the previous embodiments, the VH domain of the anti-PD-1 antibody comprises the amino acid sequence of SEQ ID NO: 14. In some embodiments of the above aspects, which may be combined with any of the previous embodiments, the VL domain of the anti-PD-1 antibody comprises the amino acid sequence of SEQ ID NO: 16. In some embodiments of the above aspects, which may be combined with any of the previous embodiments, the heavy chain of the anti-PD-1 antibody comprises the amino acid sequence of SEQ ID NO: 13, and the light chain of the anti-PD-1 antibody comprises the amino acid sequence of SEQ ID NO: 15. In some embodiments of the above aspects, which may be combined with any of the previous embodiments, the heavy chain of the anti-PD-1 antibody comprises the amino acid sequence of SEQ ID NO: 13. In some embodiments of the above aspects, which may be combined with any of the previous embodiments, the light chain of the anti-PD-1 antibody comprises the amino acid sequence of SEQ ID NO: 15. In some embodiments of the above aspects, which may be combined with any of the previous embodiments, the anti-PD-1 antibody is zimberelimab.

In some embodiments of the above aspects, which may be combined with any of the previous embodiments, the cervical cancer is recurrent or metastatic cervical cancer. In some embodiments of the above aspects, which may be combined with any of the previous embodiments, the cervical cancer has been previous treated. In a further embodiment, the cervical cancer has been previously treated with at least one of platinum-containing standard therapy. In a further embodiment, the individual received at least four cycles of an adjuvant platinum-containing regimen, and progressed or recurred within 6 months from the end of at least four cycles. In some embodiments of the above aspects, which may be combined with any of the previous embodiments, administration of the antibody-drug conjugate and anti-PD-1 antibody is second-line therapy, third-line therapy, or fourth-line therapy. In some embodiments of the above aspects, which may be combined with any of the previous embodiments, the cells of the cervical cancer express HER2. In a further embodiment, a sample obtained from the individual comprises cancer cells that express HER2 on their cell surface at a level of IHC 1+, IHC 2+, or IHC 3+. In a further embodiment, the sample obtained from the individual comprises cancer cells that express HER2 on their cell surface at a level of IHC 2+ or IHC 3+, and are positive for HER2 expression by fluorescence in situ hybridization (FISH+). In some embodiments of the above aspects, which may be combined with any of the previous embodiments, a sample obtained from the individual comprises cancer cells that express PD-L1. In some embodiments of the above aspects, which may be combined with any of the previous embodiments, the individual is not suitable for surgery or radiotherapy. In some embodiments of the above aspects, which may be combined with any of the previous embodiments, the individual is human.

In some embodiments of the above aspects, which may be combined with any of the previous embodiments, the antibody-drug conjugate is administered to the individual at a dose of about 1.5-2.0 mg/kg. In some embodiments of the above aspects, which may be combined with any of the previous embodiments, the antibody-drug conjugate is administered to the individual at a dose of about 1.5-2.0 mg/kg, wherein the dose is measured using the universal protein concentration assay method. In a further embodiment, the antibody-drug conjugate is administered to the individual at a dose of about 2.0 mg/kg. In a further embodiment, the antibody-drug conjugate is administered to the individual at a dose of about 2.0 mg/kg, wherein the dose is measured using the universal protein concentration assay method. In another embodiment, the antibody-drug conjugate is administered to the individual at a dose of 2.0 mg/kg. In another embodiment, the antibody-drug conjugate is administered to the individual at a dose of 2.0 mg/kg, wherein the dose is measured using the universal protein concentration assay method. In another embodiment, the antibody-drug conjugate is administered to the individual at a dose of about 1.5 mg/kg, wherein the dose is measured using the universal protein concentration assay method. In another embodiment, the antibody-drug conjugate is administered to the individual at a dose of about 1.5 mg/kg, wherein the dose is measured using the universal protein concentration assay method. In another embodiment, the antibody-drug conjugate is administered to the individual at a dose of 1.5 mg/kg. In another embodiment, the antibody-drug conjugate is administered to the individual at a dose of 1.5 mg/kg, wherein the dose is measured using the universal protein concentration assay method. In some embodiments of the above aspects, which may be combined with any of the previous embodiments, the dose is measured using the universal protein concentration assay method. In some embodiments of the above aspects, which may be combined with any of the previous embodiments, the antibody-drug conjugate is administered to the patient every two weeks or every 14 days. In some embodiments of the above aspects, which may be combined with any of the previous embodiments, the antibody-drug conjugate is administered to the individual at a dose of 2.0 mg/kg every two weeks or every 14 days. In some embodiments of the above aspects, which may be combined with any of the previous embodiments, the antibody-drug conjugate is administered to the individual at a dose of 1.5 mg/kg every two weeks or every 14 days. In some embodiments of the above aspects, which may be combined with any of the previous embodiments, the antibody-drug conjugate is administered intravenously to the individual. In some embodiments of the above aspects, which may be combined with any of the previous embodiments, the anti-PD-1 antibody is administered to the individual at a dose of about 240 mg. In a further embodiment, the anti-PD-1 antibody is administered to the individual at a dose of 240 mg. In some embodiments of the above aspects, which may be combined with any of the previous embodiments, the anti-PD-1 antibody is administered to the individual every two weeks or every 14 days. In some embodiments of the above aspects, which may be combined with any of the previous embodiments, the antibody-drug conjugate is disitamab vedotin administered to the individual at a dose of 2.0 mg/kg once every two weeks or 14 days, and the anti-PD-1 antibody is zimberelimab administered to the individual at a dose of 240 mg once every two weeks or 14 days. In some embodiments of the above aspects, which may be combined with any of the previous embodiments, the antibody-drug conjugate is disitamab vedotin administered to the individual at a dose of 1.5 mg/kg once every two weeks or 14 days, and the anti-PD-1 antibody is zimberelimab administered to the individual at a dose of 240 mg once every two weeks or 14 days. In some embodiments of the above aspects, which may be combined with any of the previous embodiments, administration of the antibody-drug conjugate and administration of the anti-PD-1 antibody to the individual are sequential. In a further embodiment, administration of the antibody-drug conjugate to the individual occurs prior to administration of the anti-PD-1 antibody to the individual. In another embodiment, administration of the antibody-drug conjugate to the individual occurs at least 30 minutes prior to administration of the anti-PD-1 antibody to the individual. In some embodiments of the above aspects, which may be combined with any of the previous embodiments, the antibody-drug conjugate and the anti-PD-1 antibody are administered to the individual every two weeks or 14 days for at least 6 weeks+7 days. In some embodiments of the above aspects, which may be combined with any of the previous embodiments, the antibody-drug conjugate and the anti-PD-1 antibody are administered to the individual every two weeks or 14 days for a maximum of 24 months. In some embodiments of the above aspects, which may be combined with any of the previous embodiments, administration of the antibody-drug conjugate and anti-PD-1 antibody results in a complete response (CR) or partial response (PR) in the individual. In some embodiments of the above aspects, which may be combined with any of the previous embodiments, administration of the antibody-drug conjugate and anti-PD-1 antibody results in an objective response rate (ORR) greater than 15%. In some embodiments of the above aspects, which may be combined with any of the previous embodiments, administration of the antibody-drug conjugate and anti-PD-1 antibody results in an objective response rate (ORR) greater than 25%.

In some aspects, provided herein are uses of an anti-HER2 antibody-drug conjugate for manufacture of a medicament for treating or preventing progression of cervical cancer of an individual, wherein the use comprises administering an effective amount of the anti-HER2 antibody-drug conjugate in combination with an anti-PD-1 antibody according to the method of any one of the embodiments described herein.

In some aspects, provided herein are uses of an anti-PD-1 antibody for manufacture of a medicament for treating or preventing progression of cervical cancer of an individual, wherein the use comprises administering an effective amount of the anti-PD-1 antibody in combination with an anti-HER2 antibody-drug conjugate according to the method of any one of the embodiments described herein.

In some aspects, provided herein are uses of an anti-HER2 antibody-drug conjugate and an anti-PD-1 antibody for manufacture of a medicament for treating or preventing progression of cervical cancer of an individual, wherein the use comprises administering an effective amount of the anti-HER2 antibody-drug conjugate in combination with the anti-PD-1 antibody according to the method of any one of the embodiments described herein.

In some aspects, provided herein are compositions comprising an anti-HER2 antibody-drug conjugate for use in a method of treating or preventing progression of cervical cancer in an individual, wherein the method comprises administering an effective amount of the anti-HER2 antibody-drug conjugate and an effective amount of an anti-PD-1 antibody according to the method of any one of the embodiments described herein.

In some aspects, provided herein are compositions comprising an anti-PD-1 antibody for use in a method of treating or preventing progression of cervical cancer in an individual, wherein the method comprises administering an effective amount of the anti-PD-1 antibody and an effective amount of an anti-HER2 antibody-drug conjugate according to the method of any one of the embodiments described herein.

In some aspects, provided herein are compositions comprising an anti-HER2 antibody-drug conjugate and an anti-PD-1 antibody for use in a method of treating or preventing cervical cancer in an individual, wherein the method comprising administering an effective amount of the composition according to the method of any one of the embodiments described herein.

In some aspects, provided herein are kits comprising an anti-human epidermal growth factor receptor 2 (HER2) antibody-drug conjugate and an anti-PD-1 antibody, wherein the anti-HER2 antibody-drug conjugate comprises an anti-HER2 antibody and a cytotoxic molecule, wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the anti-PD-1 antibody comprises a heavy chain comprising a heavy chain variable (VH) domain and a light chain comprising a light chain variable (VL) domain, wherein the VH domain comprises a CDR-H1 comprising the amino acid sequence STTYYWV (SEQ ID NO: 17), a CDR-H2 comprising the amino acid sequence SISYSGSTYYNPSLKS (SEQ ID NO: 18), and a CDR-H3 comprising the amino acid sequence HLGYNGRYLPFDY (SEQ ID NO: 19) and the VL domain comprises a CDR-L1 comprising the amino acid sequence TGTSSDVGFYNYVS (SEQ ID NO: 20), a CDR-L2 comprising the amino acid sequence DVSNRPS (SEQ ID NO: 21), and a CDR-L3 comprising the amino acid sequence SSYTSISTWV (SEQ ID NO: 22).

In some aspects, provided herein are kits comprising an anti-human epidermal growth factor receptor 2 (HER2) antibody-drug conjugate and an anti-PD-1 antibody, wherein the anti-HER2 antibody-drug conjugate comprises an anti-HER2 antibody and a cytotoxic molecule, wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the anti-PD-1 antibody comprises a heavy chain comprising a heavy chain variable (VH) domain and a light chain comprising a light chain variable (VL) domain, wherein the VH domain comprises a CDR-H1 comprising the amino acid sequence STTYYWV (SEQ ID NO: 17), a CDR-H2 comprising the amino acid sequence SISYSGSTYYNPSLKS (SEQ ID NO: 18), and a CDR-H3 comprising the amino acid sequence HLGYNGRYLPFDY (SEQ ID NO: 19) and the VL domain comprises a CDR-L1 comprising the amino acid sequence TGTSSDVGFYNYVS (SEQ ID NO: 20), a CDR-L2 comprising the amino acid sequence DVSNRPS (SEQ ID NO: 21), and a CDR-L3 comprising the amino acid sequence SSYTSISTWV (SEQ ID NO: 22).

In some embodiments of the above kit aspects, the kit further comprises instructions for administering an effective amount of the antibody-drug conjugate and the anti-PD-1 antibody to an individual in need thereof according to the method of any one of the embodiments described herein.

### BRIEF DESCRIPTION OF THE DRAWINGS

FIG. 1 shows the schema for a phase 2 basket study of disitamab vedotin in adult subjects with previously treated cervical cancer that express HER2. HER2-human epidermal growth factor receptor 2; Q2W=every 2 weeks or every 14 days; IHC-immunohistochemistry assay for HER2; FISH-fluorescence in situ hybridization assay for HER2.

FIG. 2 shows the schema for a single-arm, open-label, multicenter Phase 2 clinical study of disitamab vedotin in combination with zimberelimab in subjects with recurrent or metastatic cervical cancer with HER2-expression who have failed at least one line of platinum-containing standard therapy. Q2W-every 2 weeks or every 14 days; IHC=immunohistochemistry assay for HER2; DV=disitamab vedotin; ORR=objective response rate; CR=complete response; PR=partial response; DLT=dose-limiting toxicity.

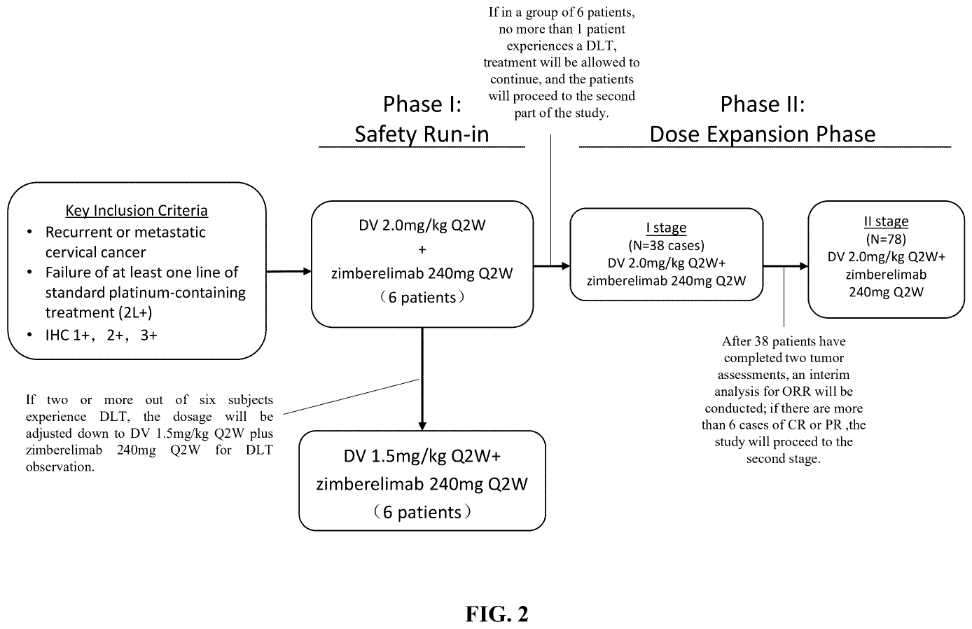

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a,” “an,” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2^{nd} ed., 2002, CRC Press; The Dictionary of Cell and Molecular Biology, 3^{rd} ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or B; A or C; B or C; A and B; A and C; B and C; A (alone); B (alone); and C (alone).

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal, or mucosal route of administration, for example, intranasally, vaginally, rectally, sublingually, or topically. Administration can also be performed, for example, once, a plurality of times, and/or over one or more extended periods.

A “cancer” refers to a broad group of various diseases characterized by the uncontrolled growth of abnormal cells in the body. A “cancer” or “cancer tissue” can include a tumor.

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “HER2” (also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP_001289938, and NP_004448. Non-limiting examples of HER2 peptide sequences are set forth in GenBank reference numbers NP OO 1005862, NP_001276865, NP_001276866, NP_001276867, and NP_004439.

The term “anti-HER2 antibody-drug conjugate” refers to an anti-HER2 antibody conjugated to a therapeutic agent (i.e., a drug or cytotoxic molecule) optionally via a linker.

An “anti-HER2 antibody”, as used herein, refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation, or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include hertuzumab, trastuzumab, pertuzumab, margetuximab, and combinations thereof.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQ C30), decreasing the dose of other medications required to treat a disease, delaying the progression of a disease, and/or prolonging survival of subjects having a disease.

The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect (e.g., diarrhea).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g., antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The phrase “pharmaceutically acceptable salt” as used herein, refers to pharmaceutically acceptable organic or inorganic salts of a compound of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, genisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-napbthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

### II. Methods of Treating Cancer

Certain aspects of the present disclosure relate to methods for treating or preventing progression of cancer (e.g., cervical cancer) in an individual, comprising administering to the individual an effective amount of an anti-human epidermal growth factor receptor 2 (HER2) antibody-drug conjugate. In some embodiments, the method further comprises administering an effective amount of an anti-PD-1 antibody to the individual. In some embodiments, the cancer is cervical cancer. In some embodiments, the cervical cancer is squamous cell carcinoma or adenocarcinoma. In some embodiments, prior to the administration of the antibody-drug conjugate, the individual has progressed during or after a first-line platinum-based standard therapy, or has progressed during or after concurrent radiotherapy and chemotherapy. In some embodiments, prior to the administration of the antibody-drug conjugate, the individual has failed first-line platinum-based standard therapy, or has failed concurrent radiotherapy and chemotherapy. In some embodiments, the cancer has previously been treated. In some embodiments, the cancer has been previously treated with at least first-line platinum-based standard therapy. In some embodiments, the cancer has been previously treated with radiotherapy.

In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the cervical cancer is selected from the group consisting of squamous cell carcinoma and adenocarcinoma.

In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the cervical cancer is selected from the group consisting of squamous cell carcinoma and adenocarcinoma.

In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule, and an effective amount of an anti-PD-1 antibody; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the anti-PD-1 antibody comprises a heavy chain comprising a heavy chain variable (VH) domain and a light chain comprising a light chain variable (VL) domain, wherein the VH domain comprises a CDR-H1 comprising the amino acid sequence STTYYWV (SEQ ID NO: 17), a CDR-H2 comprising the amino acid sequence SISYSGSTYYNPSLKS (SEQ ID NO: 18), and a CDR-H3 comprising the amino acid sequence HLGYNGRYLPFDY (SEQ ID NO: 19) and the VL domain comprises a CDR-L1 comprising the amino acid sequence TGTSSDVGFYNYVS (SEQ ID NO: 20), a CDR-L2 comprising the amino acid sequence DVSNRPS (SEQ ID NO: 21), and a CDR-L3 comprising the amino acid sequence SSYTSISTWV (SEQ ID NO: 22). In some aspects, provided herein are methods for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule, and an effective amount of an anti-PD-1 antibody; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and wherein the anti-PD-1 antibody comprises a heavy chain comprising a heavy chain variable (VH) domain and a light chain comprising a light chain variable (VL) domain, wherein the VH domain comprises a CDR-H1 comprising the amino acid sequence STTYYWV (SEQ ID NO: 17), a CDR-H2 comprising the amino acid sequence SISYSGSTYYNPSLKS (SEQ ID NO: 18), and a CDR-H3 comprising the amino acid sequence HLGYNGRYLPFDY (SEQ ID NO: 19) and the VL domain comprises a CDR-L1 comprising the amino acid sequence TGTSSDVGFYNYVS (SEQ ID NO: 20), a CDR-L2 comprising the amino acid sequence DVSNRPS (SEQ ID NO: 21), and a CDR-L3 comprising the amino acid sequence SSYTSISTWV (SEQ ID NO: 22).

In some embodiments of the above aspects, the VH domain of the anti-HER2 antibody comprises the amino acid sequence of SEQ ID NO: 7, and the VL domain of the anti-HER2 antibody comprises the amino acid sequence of SEQ ID NO: 8. In some embodiments of the above aspects, the VH domain of the anti-HER2 antibody comprises the amino acid sequence of SEQ ID NO: 7. In some embodiments of the above aspects, which may be combined with any of the previous embodiments, the VL domain of the anti-HER2 antibody comprises the amino acid sequence of SEQ ID NO: 8. In some embodiments of the above aspects, which may be combined with any of the previous embodiments, the heavy chain of the anti-HER2 antibody comprises the amino acid sequence of SEQ ID NO: 9 or SEQ ID NO: 10, and the light chain of the anti-HER2 antibody comprises the amino acid sequence of SEQ ID NO: 11. In some embodiments of the above aspects, which may be combined with any of the previous embodiments, the heavy chain of the anti-HER2 antibody comprises the amino acid sequence of SEQ ID NO: 9 or SEQ ID NO: 10. In some embodiments of the above aspects, which may be combined with any of the previous embodiments, the light chain of the anti-HER2 antibody comprises the amino acid sequence of SEQ ID NO: 11. In some embodiments of the above aspects, which may be combined with any of the previous embodiments, the cytotoxic molecule comprises a tubulin inhibitor or DNA damaging agent. In a further embodiment of the above aspects, the cytotoxic molecule comprises a tubulin inhibitor, wherein the tubulin inhibitor comprises a dolastatin or derivative thereof, an auristatin or derivative thereof, or a maytansinoid or derivative thereof. In another embodiment of the above aspects, the tubulin inhibitor comprises monomethyl auristatin E (MMAE), monomethyl auristatin F (MMAF), or auristatin F (AF). In an additional embodiment of the above aspects, the tubulin inhibitor comprises emtansine (DM1), maytansine (DM3), or ravtansine (DM4). In yet another embodiment, the tubulin inhibitor comprises emtansine (DM1), maytansine (DM3), or ravtansine (DM4). In some embodiments of the above aspects, which may be combined with any of the previous embodiments, the cytotoxic molecule comprises an amanitin, anthracycline, baccatin, camptothecin, cemadotin, colchicine, colcemid, combretastatin, cryptophycin, discodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid. In some embodiments of the above aspects, which may be combined with any of the previous embodiments, the antibody-drug conjugate is represented by formula Ab-(L-U) n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. In a further embodiment of the above aspects, the linker is attached to the anti-HER2 antibody via a thiol or amino moiety. In some embodiments of the above aspects, which may be combined with any of the previous embodiments, the linker is selected from the group consisting of maleimidocaproyl valine citrulline p-amino-benzyl (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl) propanamido) ethoxy) ethoxy) propanoate), Mal-di-EG-OSu (2,5-dioxopyrrolidin-1-yl 3-(2-(2-(2,5-dioxo-2,5-dihydro-1H-pyrrol-1-yl) ethoxy) ethoxy) propanoate), Mal-Tri-EG-OSu (2,5-dioxopyrrolidin-1-yl 3-(2-(2-(2-(2,5-dioxo-2,5-dihydro-1H-pyrrol-1-yl) ethoxy) ethoxy) ethoxy) ethoxypropanoate), Mal-Tetra-EG-OSu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-OSu (2,5-dioxopyrrolidin-1-yl 3 (2-(2-(2-bromoacetamido) ethoxy) ethoxy) propanoate), Py-ds-prp-OSu (2-5-dioxopyrrolidin-1-yl 3-(pyridine-2-yldisulfanyl) propanoate), Py-ds-Prp-OPEP (perfluorophenyl 3-(pyridine-2-yldisulfanyl) propanoate), Py-ds-dmBut-OSu (2,5-dioxopyrrolidin-1-yl 4-methyl-4-(pyridine-2-yldisulfanyl) pentanoate, Py-ds-dmBut-OPF (perfluorophenyl 4-methyl-4-(pyridine-2-yldisulfanyl) pentanoate), SMCC (N-succinimidyl 4-(maleimidomethyl)cyclohexanecarboxylate), MBS (3-maleimidobenzoic acid N-hydroxysuccinimide ester), SATA (S—(N-succinimidyl) thioacetate), SPDP ((N-succinimidyl 3-(2-pyridyldithio) propionate), and SMPT ((N-succinimidyloxy carbonyl)-1-methyl-1-(2-pyridyldithio) toluene). In some embodiments of the above aspects, which may be combined with any of the previous embodiments, the antibody-drug conjugate is disitamab vedotin.

### Antibody-Drug Conjugates

In some embodiments, the anti-HER2 antibody comprises a heavy chain comprising a heavy chain variable (VH) domain and a light chain comprising a light chain variable (VL) domain. In some embodiments, the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and/or the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6). In some embodiments, the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and/or the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6). In some embodiments, the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6). In some embodiments, the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6).

In some embodiments, the anti-HER2 antibody comprises a VH domain that comprises the amino acid sequence of SEQ ID NO: 7 and/or a VL domain that comprises the amino acid sequence of SEQ ID NO:8. In some embodiments, the anti-HER2 antibody comprises a VH domain that comprises the amino acid sequence of SEQ ID NO:7 and a VL domain that comprises the amino acid sequence of SEQ ID NO: 8.

In some embodiments, the anti-HER2 antibody comprises a heavy chain that comprises the amino acid sequence of SEQ ID NO: 9 or SEQ ID NO: 10 and/or a light chain that comprises the amino acid sequence of SEQ ID NO: 11. In some embodiments, the anti-HER2 antibody comprises a heavy chain that comprises the amino acid sequence of SEQ ID NO: 9 and/or a light chain that comprises the amino acid sequence of SEQ ID NO: 11. In some embodiments, the anti-HER2 antibody comprises a heavy chain that comprises the amino acid sequence of SEQ ID NO: 10 and/or a light chain that comprises the amino acid sequence of SEQ ID NO: 11. In some embodiments, the anti-HER2 antibody comprises a heavy chain that comprises the amino acid sequence of SEQ ID NO: 9 or SEQ ID NO: 10 and a light chain that comprises the amino acid sequence of SEQ ID NO: 11. In some embodiments, the anti-HER2 antibody comprises a heavy chain that comprises the amino acid sequence of SEQ ID NO: 9 and a light chain that comprises the amino acid sequence of SEQ ID NO: 11. In some embodiments, the anti-HER2 antibody comprises a heavy chain that comprises the amino acid sequence of SEQ ID NO: 10 and a light chain that comprises the amino acid sequence of SEQ ID NO: 11.

In some embodiments, the anti-HER2 antibody comprises one, two, three, four, five, or six CDR sequences shown in Table 1 below. In some embodiments, the anti-HER2 antibody comprises one, two, or all three CDR sequences from a VH domain or heavy chain sequence shown in Table 1 below and/or one, two, or all three CDR sequences from a VL domain or light chain sequence shown in Table 1 below. In some embodiments, the anti-HER2 antibody comprises a VH domain sequence and/or VL domain sequence shown in Table 1 below. In some embodiments, the anti-HER2 antibody comprises a heavy chain and/or a light chain sequence shown in Table 1 below.

TABLE 1
Anti-HER2 antibody sequences

| Description | Amino acid sequence | SEQ ID NO |
|---|---|---|
| disitamab | DYYIH | 1 |
| CDR-H1 | | |
| disitamab | RVNPDHGDSYYNQKFKD | 2 |
| CDR-H2 | | |
| disitamab | NYLFDH | 3 |
| CDR-H3 | | |
| disitamab | KASQDVGTAVA | 4 |
| CDR-L1 | | |
| disitamab | WASIRHT | 5 |
| CDR-L2 | | |
| disitamab | HQFATYT | 6 |
| CDR-L3 | | |
| disitamab | EVQLVQSGAEVKKPGATVKISCKVSGYTFTDYYIHWVQQA | 7 |
| VH domain | PGKGLEWMGRVNPDHGDSYYNQKFKDKATITADKSTDTA | |
| | YMELSSLRSEDTAVYFCARNYLFDHWGQGTLVTVSS | |
| disitamab | DIQMTQSPSSVSASVGDRVTITCKASQDVGTAVAWYQQKP | 8 |
| VL domain | GKAPKLLIYWASIRHTGVPSRFSGSGSGTDFTLTISSLQPEDF | |
| | ATYYCHQFATYTFGGGTKVEIK | |
| disitamab | EVQLVQSGAEVKKPGATVKISCKVSGYTFTDYYIHWVQQA | 9 |
| heavy chain | PGKGLEWMGRVNPDHGDSYYNQKFKDKATITADKSTDTA | |
| sequence 1 | YMELSSLRSEDTAVYFCARNYLFDHWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKKVEPKSCDKTHTCPPCPAPELLGGPSVFLFPPK | |
| | PKDTLMISRTPEVTCVVVDVSHEDPEVKFNWYVDGVEVHN | |
| | AKTKPREEQYNSTYRVVSVLTVLHQDWLNGKEYKCKVSN | |
| | KALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVFSCSVMHEALHNHYTQKSLSLSPG | |
| disitamab | EVQLVQSGAEVKKPGATVKISCKVSGYTFTDYYIHWVQQA | 10 |
| heavy chain | PGKGLEWMGRVNPDHGDSYYNQKFKDKATITADKSTDTA | |
| sequence 2 | YMELSSLRSEDTAVYFCARNYLFDHWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKKVEPKSCDKTHTCPPCPAPELLGGPSVFLFPPK | |
| | PKDTLMISRTPEVTCVVVDVSHEDPEVKFNWYVDGVEVHN | |
| | AKTKPREEQYNSTYRVVSVLTVLHQDWLNGKEYKCKVSN | |
| | KALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVFSCSVMHEALHNHYTQKSLSLSPGK | |
| disitamab | DIQMTQSPSSVSASVGDRVTITCKASQDVGTAVAWYQQKP | 11 |
| light chain | GKAPKLLIYWASIRHTGVPSRFSGSGSGTDFTLTISSLQPEDF | |
| | ATYYCHQFATYTFGGGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| disitamab | ARNYLFDHW | 12 |
| CDR-H3 | | |
| (long) | | |

In some embodiments, the anti-HER2 antibody is disitamab (RC48). See, e.g., U.S. Pat. No. 10,087,260, the contents of which are incorporated herein by reference in its entirety.

In some embodiments, the cytotoxic molecule of an antibody-drug conjugate of the present disclosure comprises a tubulin inhibitor or DNA damaging agent.

In some embodiments, the tubulin inhibitor comprises a dolastatin or derivative thereof, auristatin or derivative thereof, or maytansinoid or derivative thereof. In some embodiments, the tubulin inhibitor comprises monomethyl auristatin E (MMAE), monomethyl auristatin F (MMAF), or auristatin F (AF). In some embodiments, the tubulin inhibitor comprises mertansine (DM1), maytansine (DM3), or ravtansine (DM4). Structures for MMAE and MMAF are provided below. Additional descriptions and examples of tubulin inhibitors may be found, e.g., in Chen, H. et al. (2017) Molecules 22 (8): 1281.

In some embodiments, the DNA damaging agent comprises a calicheamicin, duocarmycin, pyrrolobenzodiazepine (PBD), or SN-38.

In some embodiments, the cytotoxic molecule comprises an amanitin, anthracycline, baccatin, camptothecin, cemadotin, colchicine, colcemid, combretastatin, cryptophycin, discodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid.

In some embodiments, the antibody-drug conjugate is represented by formula Ab-(L-U) n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. For example, in some embodiments, the antibody-drug conjugate used is named RC48-mc-vc-pAB-MMAE, which conforms to the structure of the general formula Ab-(L-U) n, in which RC48 (a humanized anti-HER2 monoclonal antibody) is coupled to MMAE through the linker mc-vc-pAB, and the number of coupling ranges from 1 to 8, including 1, 2, 3, 4, 5, 6, 7, 8 or a combination of antibody-drug conjugates with varying MMAE coupling numbers ranging from 1 to 8. In some embodiments, the number of cytotoxic molecules bound to the antibody is given as an average number of cytotoxic molecules bound to the antibody, e.g., within a given sample, population, or composition. In some embodiments, an average of 4 MMAE molecules are conjugated to the antibody, e.g., via a linker such as mc-vc-pAB.

In some embodiments, the linker is attached to the anti-HER2 antibody via a thiol or amino moiety. In some embodiments, the cytotoxic molecule is conjugated to the antibody through site-directed or undirected conjugation.

In some embodiments, the linker is selected from the group consisting of maleimidocaproyl valine citrulline p-amino-benzyl (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl) propanamido) ethoxy) ethoxy) propanoate), Mal-di-EG-Osu (2,5-dioxopyrrolidin-1-yl 3-(2-(2-(2,5-dioxo-2,5-dihydro-1H-pyrrol-1-yl) ethoxy) ethoxy) propanoate), Mal-Tri-EG-Osu (2,5-dioxopyrrolidin-1-yl 3-(2-(2-(2-(2,5-dioxo-2,5-dihydro-1H-pyrrol-1-yl) ethoxy) ethoxy) ethoxy) ethoxypropanoate), Mal-Tetra-EG-Osu (2,5-dioxopyrrolidin-1-yl 1-(2,5-dioxo-2,5-dihydro-1H-pyrrol-1-yl)-3-oxo-7,10,13, 16-tetraoxa-4-azanonadecan-19-oate), Br-di-EG-Osu (2,5-dioxopyrrolidin-1-yl 3 (2-(2-(2-bromoacetamido) ethoxy) ethoxy) propanoate), Py-ds-prp-Osu (2-5-dioxopyrrolidin-1-yl 3-(pyridine-2-yldisulfanyl) propanoate), Py-ds-Prp-OPEP (perfluorophenyl 3-(pyridine-2-yldisulfanyl) propanoate), Py-ds-dmBut-Osu (2,5-dioxopyrrolidin-1-yl 4-methyl-4-(pyridine-2-yldisulfanyl) pentanoate, Py-ds-dmBut-OPF (perfluorophenyl 4-methyl-4-(pyridine-2-yldisulfanyl) pentanoate), SMCC (N-succinimidyl 4-(maleimidomethyl) cyclohexanecarboxylate), MBS (3-maleimidobenzoic acid N-hydroxysuccinimide ester), SATA (S—(N-succinimidyl) thioacetate), SPDP ((N-succinimidyl 3-(2-pyridyldithio) propionate), and SMPT ((N-succinimidyloxy carbonyl)-1-methyl-1-(2-pyridyldithio) toluene).

In some embodiments, the linker is a linker described in Table 2 below.

TABLE 2
Exemplary linkers for antibody-drug conjugates.

| Abbreviation | Full name | Feature |
|---|---|---|
| mc | Maleimidocaproyl | Non-cleavable |
| mc-vc-pAB | Maleimidocaproyl valine citrulline p-amino-benzyl | Cleavable |
| 3-MPA | 3-maleimido-propionic acid | Non-cleavable |
| Mal-di-EG-OPFP | Perfluorophenyl | Non-cleavable |
| | 3-(2-(2-(3-(2,5-dioxo-2,5-dihydro-1H-pyrrol-1- | |
| | yl)propanamido)ethoxy) ethoxy)propanoate | |
| Mal-di-EG-Osu | 2,5-dioxopyrrolidin-1-yl | Non-cleavable |
| | 3-(2-(2-(2,5-dioxo-2,5-dihydro-1H-pyrrol-1- | |
| | yl)ethoxy)ethoxy) propanoate | |
| Mal-Tri-EG-Osu | 2,5-dioxopyrrolidin-1-yl | Non-cleavable |
| | 3-(2-(2-(2-(2,5-dioxo-2,5-dihydro-1H-pyrrol-1- | |
| | yl)ethoxy)ethoxy)ethoxy) propanoate | |
| Mal-Tetra-EG- | 2,5-dioxopyrrolidin-1-yl | Non-cleavable |
| Osu | 1-(2,5-dioxo-2,5-dihydro-1H-pyrrol-1-yl)-3-oxo- | |
| | 7,10,13,16-tetraoxa-4-azanonadecan-19-oate | |
| Br-di-EG-Osu | 2,5-dioxopyrrolidin-1-yl | Non-cleavable |
| | 3-(2-(2-(2-bromoacetamido)ethoxy)ethoxy)propanoate | |
| Py-ds-prp-Osu | 2,5-dioxopyrrolidin-1-yl 3-(escrye-2- | Reducible |
| | yldisulfanyl)propanoate | |
| Py-ds-Prp-OPFP | Perfluorophenyl 3-(escrye-2-yldisulfanyl)propanoate | Reducible |
| Py-ds-dmBut-Osu | 2,5-dioxopyrrolidin-1-yl 4-methyl-4-(escrye-2-yldisulfanyl) | Reducible |
| | pentanoate | |
| Py-ds-dmBut- | Perfluorophenyl 4-methyl-4-(escrye-2- | Reducible |
| OPF | yldisulfanyl)pentanoate | |
| SMCC | N-succinimidyl 4-(maleimidomethyl) | Non-cleavable |
| | cyclohexanecarboxylate | |
| MBS | 3-maleimidobenzoic acid N-hydroxysuccinimide ester | Non-cleavable |
| SATA | S-(N-succinimidyl) thioacetate | Non-cleavable |
| SPDP | N-succinimidyl 3-(2-pyridyldithio)propionate | Reducible |
| SMPT | (N-succinimidyl carbonyl)-1-methyl-1-(2-pyridyldithio) | Reducible |
| | toluene | |

In some embodiments, the anti-HER2 antibody-drug conjugate is disitamab vedotin (DV; also referred to as RC48-ADC). Disitamab vedotin (DV hereafter) is an antibody-drug conjugate (ADC) that targets cancers expressing HER2, an oncogenic growth factor receptor which promotes cell proliferation and survival. DV consists of an anti-HER2 monoclonal antibody disitamab (RC48) conjugated with an average of 4 molecules of the tubulin-disrupting anti-mitotic agent monomethyl auristatin E (MMAE) via a cleavable peptide linker. See, e.g., U.S. Pat. No. 10,087,260 and U.S. PG Pub. Nos. 2020/0289663 and 2021/0154314, the contents of which are incorporated herein by reference in their entirety. DV has multimodal antitumor mechanisms of action that include direct cytotoxicity of HER2-expressing cancer cells and bystander effect based-cytotoxicity of neighboring cells, both of which are mediated by the intracellular release of MMAE within the targeted cell. Released MMAE can induce immunogenic cell death (ICD), which promotes immune cell recruitment to the tumor. In addition, DV stimulates Fc-gamma receptor mediated antibody-dependent cellular cytotoxicity (ADCC), which can lead to target cell death. DV also inhibits HER2-activated downstream signaling pathways, further blocking cellular growth and proliferation.

### Anti-PD-1 Antibodies

Programmed death-1 (PD-1) is an inhibitory receptor expressed on T-cells that promotes apoptosis of inflammatory T-cells and inhibits apoptosis of anti-inflammatory regulatory T-cells, thereby promoting self-tolerance and preventing autoimmune diseases. To evade immune surveillance, tumor cells often exploit this system by over-expressing PD-L1, the ligand which binds to and activates PD-1. Anti-PD-1 antibodies are used to block the binding of PD-L1 to PD-1 and thereby enhance immune response against cancer cells. Anti-PD-1 antibodies have showed promising results in various cancers via enhancing T cell functions. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116.

In some embodiments, the anti-PD-1 antibody is a human antibody, a humanized antibody, or a chimeric antibody. In some embodiments, the anti-PD-1 antibody is one or more of zimberelimab, toripalimab, MDX-1 106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, ENUM 388D4, cemiplimab, or dostarlimab. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings).

In some embodiments, the PD-1 antibody comprises one or more of zimberelimab, toripalimab, nivolumab, pembrolizumab, cemiplimab, retifanlimab, or dostarlimab. In some embodiments, the anti-PD-1 antibody is zimberelimab.

Zimberelimab (INN: 11413), also known as AB122, GLS-010, and WBP-3055, refers to a human immunoglobulin G4 (IgG4) monoclonal antibody directed against the negative immunoregulatory human cell receptor programmed cell death protein 1 (PD-1; PDCD1; CD279). Without being bound by theory, zimberelimab targets, binds to and inhibits PD-1 and its downstream signaling pathways, which restores immune function through the activation of T cells and T-cell-mediated immune responses against tumor cells. In some embodiments, the anti-PD-1 antibody is zimberelimab.

In some embodiments, the anti-PD-1 antibody comprises one, two, three, four, five, or six CDR sequences shown in Table 3 below. In some embodiments, the anti-PD-1 antibody comprises one, two, or all three CDR sequences from a VH domain or heavy chain sequence shown in Table 3 below and/or one, two, or all three CDR sequences from a VL domain or light chain sequence shown in Table 3 below. In some embodiments, the anti-PD-1 antibody comprises a VH domain sequence and/or VL domain sequence shown in Table 3 below. In some embodiments, the anti-PD-1 antibody comprises a heavy chain and/or a light chain sequence shown in Table 3 below.

TABLE 3
Anti-PD-1 antibody sequences

| Description | Amino acid sequence | SEQ ID NO |
|---|---|---|
| zimberelimab | QLQLQESGPG LVKPSETLTL TCTVSADSIS | 13 |
| heavy chain | STTYYWVWIR QPPGKGLEWI GSISYSGSTY | |
| | YNPSLKSRVT VSVDTSKNQF SLKLNSVAAT | |
| | DTALYYCARH LGYNGRYLPF DYWGQGTLVT | |
| | VSSASTKGPS VFPLAPCSRS TSESTAALGC | |
| | LVKDYFPEPV TVSWNSGALT SGVHTFPAVL | |
| | QSSGLYSLSS VVTVPSSSLG TKTYTCNVDH | |
| | KPSNTKVDKR VESKYGPPCP PCPAPEFLGG | |
| | PSVFLFPPKP KDTLMISRTP EVTCVVVDVS | |
| | QEDPEVQFNW YVDGVEVHNA KTKPREEQFN | |
| | STYRVVSVLT VLHQDWLNGK EYKCKVSNKG | |
| | LPSSIEKTIS KAKGQPREPQ VYTLPPSQEE | |
| | MTKNQVSLTC LVKGFYPSDI AVEWESNGQP | |
| | ENNYKTTPPV LDSDGSFFLY SRLTVDKSRW | |
| | QEGNVFSCSV MHEALHNHYT QKSLSLSLGK | |
| zimberelimab | QLQLQESGPGLVKPSETLTLTCTVSADSISSTTYYWVWIR | 14 |
| VH domain | QPPGKGLEWIGSISYSGSTYYNPSLKSRVTVSVDTSKNQF | |
| | SLKLNSVAATDTALYYCARHLGYNGRYLPFDYWGQGTL | |
| | VTVSS | |
| zimberelimab | QSALTQPASV SGSPGQSITI SCTGTSSDVG | 15 |
| light chain | FYNYVSWYQQ HPGKAPELMI YDVSNRPSGV | |
| | SDRFSGSKSG NTASLTISGL QAEDEADYYC | |
| | SSYTSISTWV FGGGTKLTVL GQPKAAPSVT | |
| | LFPPSSEELQ ANKATLVCLI SDFYPGAVTV | |
| | AWKADSSPVK AGVETTTPSK QSNNKYAASS | |
| | YLSLTPEQWK SHRSYSCQVT HEGSTVEKTV | |
| | APTECS | |
| zimberelimab | QSALTQPASVSGSPGQSITISCTGTSSDVGFYNYVSWYQQ | 16 |
| VL domain | HPGKAPELMIYDVSNRPSGVSDRFSGSKSGNTASLTISGL | |
| | QAEDEADYYCSSYTSISTWVFGGGTKLTVLGQP | |
| zimberelimab | STTYYWV | 17 |
| CDR-H1 | | |
| zimberelimab | SISYSGSTYYNPSLKS | 18 |
| CDR-H2 | | |
| zimberelimab | HLGYNGRYLPFDY | 19 |
| CDR-H3 | | |
| zimberelimab | TGTSSDVGFYNYVS | 20 |
| CDR-L1 | | |
| zimberelimab | DVSNRPS | 21 |
| CDR-L2 | | |
| zimberelimab | SSYTSISTWV | 22 |
| CDR-L3 | | |

In some embodiments, zimberelimab is administered in a pharmaceutical composition comprising zimberelimab and one or more pharmaceutically acceptable carriers. The pharmaceutical compositions are typically administered as an intravenous infusion. In some embodiments, zimberelimab is administered intravenously.

In some embodiments, the VH domain of the anti-PD-1 antibody comprises the amino acid sequence of SEQ ID NO: 14, and the VL domain of the anti-PD-1 antibody comprises the amino acid sequence of SEQ ID NO: 16. In some embodiments, the VH domain of the anti-PD-1 antibody comprises the amino acid sequence of SEQ ID NO: 14. In some embodiments, the VL domain of the anti-PD-1 antibody comprises the amino acid sequence of SEQ ID NO: 16. In some embodiments, the heavy chain of the anti-PD-1 antibody comprises the amino acid sequence of SEQ ID NO: 13, and the light chain of the anti-PD-1 antibody comprises the amino acid sequence of SEQ ID NO: 15. In some embodiments, the heavy chain of the anti-PD-1 antibody comprises the amino acid sequence of SEQ ID NO: 13. In some embodiments, the light chain of the anti-PD-1 antibody comprises the amino acid sequence of SEQ ID NO: 15. In some embodiments, the anti-PD-1 antibody is zimberelimab.

### Cancers to be Treated

In some embodiments, the cancer is cervical cancer. In some embodiments, the cervical cancer is selected from the group consisting of squamous cell carcinoma and adenocarcinoma. In some embodiments, the cervical cancer is squamous cell carcinoma. In some embodiments, the cervical cancer is adenocarcinoma. In some embodiments, the cancer is recurrent or metastatic. In some embodiments, the cancer is recurrent. In some embodiments, the cancer is metastatic. In some embodiments, the cancer has previously been treated. In some embodiments, the cancer has been previously treated with at least first-line platinum-based therapy. In some embodiments, the cancer has been previously treated with radiotherapy. In some embodiments, the administration of the antibody-drug conjugate according to the methods of the present disclosure is a second-line therapy, third-line therapy, fourth-line therapy, or post-fourth-line therapy. In some embodiments, the individual is not a candidate for surgery or radiotherapy.

In some embodiments, cells of the cancer express HER2. In some embodiments, the cancer is a HER2-positive (HER2+) cancer. In some embodiments, cells of the cancer exhibit HER2 gene amplification. In some embodiments, cells of the cancer overexpress HER2, e.g., on their cell surface. Overexpression and/or amplification of HER2 is seen in many malignancies including cervical, breast, gastric, ovarian, pancreatic, colorectal, and endometrial cancer (Neve, R. M. et al. (2001) Ann Oncol 12 (Suppl1): S9-S13; Menard, S. et al. (2003) Oncogene 22 (42): 6570-6578; Moasser, M. M. (2007) Oncogene 26 (45): 6469-6487; Iqbal, N. et al. (2014) Mol Biol Int 2014:852748).

In some embodiments, a sample obtained from the individual comprises cancer cells that express HER2 on their cell surface, as measured by an immunohistochemistry (IHC) assay. IHC assays for HER2 involve semi-quantitative measurements of HER2 cellular expression/overexpression. These are typically done by staining a sample including cancer cells using a primary anti-HER2 antibody, followed by visualization using a labeled secondary antibody and subsequent analysis of HER2 expression, e.g., membrane expression on the surface. Variables factoring into the IHC score (given on a scale from 0 to 3+) can include intensity of staining, location and/or completeness of staining (e.g., surface/membrane expression), and proportion of tumor cells exhibiting staining. Methods and criteria for determining HER2 status by IHC are known in the art and can be found, e.g., in Wolff, A. C. et al. (2013) J Clin Oncol 31 (31): 3997-4013 and Wolff, A. C. et al. (2018) J Clin Oncol 36 (20): 2105-2122. See also the HercepTest™ semi-quantitative IHC test kit.

Evaluation of HER2 by IHC may involve one or more, or all, of the steps of:
1. The entire section is first observed under low magnification to determine whether the staining is satisfactory and whether there is heterogeneity in HER2 expression;
2. Quality control slides are read when evaluating; cytoplasmic and nuclear staining should be negligible, and normal epithelium should not show strong cell membrane staining;
3. Tissue margins and poorly prepared (e.g., obviously extruded) cancer tissue is ignored during evaluation.
4. If the tumor has obvious heterogeneity, the percentage of each scoring level is indicated separately when interpreting.
5. If invasive cancer is the object during evaluation, it is indicated separately if the non-invasive cancer part has overexpressed HER2 (2+ or 3+).
6. If multiple blocks or sections are detected, results are reported separately.

In some embodiments, a sample comprising cancer cells obtained from an individual is assayed for HER2 gene amplification or gene expression by an in situ hybridization (ISH) assay. In some embodiments, the in situ hybridization assay is a fluorescence in situ hybridization (FISH) assay. In some embodiments, a sample obtained from the individual comprises cancer cells that exhibit HER2 gene amplification, as measured by an in situ hybridization (ISH) assay (ISH-positive). In some embodiments, a sample obtained from the individual comprises cancer cells that exhibit HER2 gene amplification, as measured by a fluorescence in situ hybridization (FISH) assay (FISH-positive, or FISH+). In some embodiments, a sample obtained from the individual comprises cancer cells that do not exhibit HER2 gene amplification, as measured by an in situ hybridization (ISH) assay (ISH-negative). In some embodiments, a sample obtained from the individual comprises cancer cells that do not exhibit HER2 gene amplification, as measured by a fluorescence in situ hybridization (FISH) assay (FISH-negative, or FISH−). ISH assays for HER2 gene amplification typically involve measurement of level of hybridization to a HER2-specific probe using microscopy. In some embodiments, cells are stained with dual probes: a HER2-specific probe, and a control probe (hybridizing to, e.g., chromosome 17 or CEP17), such that the ratio of HER2: control signal is indicative of HER2 copy number and/or amplification. Variables factoring into the ISH status can include number of signal copies of HER2, ratio of HER2: control copy number, and formation of HER2 clusters. In some embodiments, HER2 is assessed by FISH using dual probes, e.g., using HER2 and CEP17 probes. Methods and criteria for determining HER2 amplification status by ISH are known in the art and can be found, e.g., in Wolff, A. C. et al. (2013) J Clin Oncol 31 (31): 3997-4013 and Wolff, A. C. et al. (2018) J Clin Oncol 36 (20): 2105-2122. Detection of HER2 may be performed by FISH, e.g., using one or more, or all, of the following steps:
(1) Selecting a representative wax block of tumor tissue. Section by professional and technical personnel, the section is complete, smooth, of uniform thickness, without affecting the diagnosis of knife mark wrinkles. (Tissue containing calcified particles and other uncontrollable factors are excluded), section thickness: 4-5 μm;
(2) Tissue section pretreatment using either of the following methods:

Method 1 (Manual operation):
a) Immerse in xylene and dewaxed twice, 15 minutes each time, and then immerse in 100% ethanol for 5 minutes at room temperature,
b) Rehydrate in 100% ethanol, 85% ethanol and 70% ethanol for 2 minutes respectively at room temperature, then immerse in deionized water at room temperature for 3 minutes,
c) Treatment with 90˜93° C. deionized water for 20 minutes,
d) 1 ml gastric enzyme storage solution (200 mg/mL) is dissolved in 200 ml 0.01MHCL to obtain gastric enzyme working solution (1 mg/ml); Soak the tissue section in gastric enzyme working solution and incubate at 37° C. for 15-30 minutes (the time depends on the thickness of the tissue, generally about 20 minutes),
e) After digestion by gastric enzymes, then rinse in deionized water for 5 minutes,
f) Dehydrate respectively in 70% ethanol, 85% ethanol and 100% ethanol for 2 minutes at room temperature,
g) After drying, then perform the following hybrid denaturation.

Method 1 (Fully automatic):
a) Soak in xylene for dewaxing twice at room temperature for 15 minutes each, and then immerse in 100% ethanol twice for 5 minutes each,
b) Dry tissue section at room temperature,
c) Initialize the system and select program, fill the reagent according to the instrument algorithm,
d) Place the dry slides tissue face upward on the glass shelf, put it in the reaction tank, cover the reaction tank cover, close the machine cover, and run the selected program;

(3) Hybridization apparatus denatured hybridization using the following steps:
a) Drop 10 μL probe mixture into the slide hybridization area, immediately cover the slide and seal the edge with rubber glue,
b) Prepare hybridization machine, covariance condition: 75° C., 5 minutes, hybridization condition: 37° C., 16 h; (be careful to maintain humidity in hybridization instrument);

(4) Glass slide rinsing (need to avoid light operation) using the following steps:
a) Carefully remove the cover glass slide, place the glass slide in a solution of 0.3% NP-40/2×SSC at 73° C., shake for 1˜3 seconds, wash for 2 minutes,
b) Rinse at room temperature in 70% ethanol for 3 minutes;

(5) Counterstaining using the following steps:
a) Naturally dried glass slides in dark;
b) Drop 10 μL DAPI at the hybridization site and immediately cover the cover glass. Put in the dark for 10 to 20 minutes, then observe the glass slides under fluorescence microscope with appropriate filter group.

Assessment of HER2, e.g., in a FISH section, for example, generated as described above, may be performed using any suitable method known in the art. For example, using one or more, or all of the following steps:
(a) Observe whole FISH section under low magnification to preliminarily determine the test quality (such as the normal cell signals of normal tissues in the specimen) and whether there is heterogeneity in HER2 amplification;
(b) Find at least 2 areas of invasive cancer and count at least 20 invasive cancer cells. FISH is not suitable for microinvasive nidus with too few cells;
(c) IHC sections can be used to determine the areas of invasive cancer that may be amplified; and
(d) Observe HER2 and CEP17 signals through a specific channel filter under high magnification (60× or 100× objective), and calculate the signal count and ratio.

In some embodiments, the sample is a biopsy sample, e.g., from a core needle biopsy. In some embodiments, the sample is from an incisional or excisional surgical procedure. In some embodiments, the sample is a formalin-fixed paraffin embedded (FFPE) tissue block or sample, e.g., with corresponding hematoxylin and eosin (H&E) stain. In some embodiments, the sample comprises unstained slides sectioned from a tissue block, e.g., a FFPE tissue block. In some embodiments, the sample is from a primary tumor or metastasis, e.g., from the chest wall, regional lymph node, or a distant organ. In some embodiments, the same sample is used for IHC and ISH assays. In some embodiments, different samples from the same individual are used for IHC and ISH assays. In some embodiments, the same sample is used for IHC and FISH assays. In some embodiments, different samples from the same individual are used for IHC and FISH assays.

In some embodiments, the cancer is a HER2-low cancer. In some embodiments, a sample obtained from the individual (e.g., from the cancer of the individual) comprises cancer cells that express HER2 on their cell surface at a level of IHC1+, as measured by IHC assay (IHC1+). In some embodiments, a sample obtained from the individual (e.g., from the cancer of the individual) comprises cancer cells that express HER2 on their cell surface at a level of IHC2+, as measured by IHC assay (IHC2+); and a sample obtained from the individual comprises cancer cells that do not exhibit HER2 gene amplification, as measured by ISH assay (IHC2+/ISH−). In some embodiments, a sample obtained from the individual (e.g., from the cancer of the individual) comprises cancer cells that express HER2 on their cell surface at a level of IHC2+, as measured by IHC assay (IHC2+); and a sample obtained from the individual comprises cancer cells that do not exhibit HER2 gene amplification, as measured by FISH assay (IHC2+/FISH−). In some embodiments, a sample obtained from the individual (e.g., from the cancer of the individual) is IHC1+. In some embodiments, a sample obtained from the individual (e.g., from the cancer of the individual) is IHC1+/ISH−. In some embodiments, a sample obtained from the individual (e.g., from the cancer of the individual) is IHC1+/FISH−.

In some embodiments, the cancer is a HER2-positive cancer. In some embodiments, a sample obtained from the individual (e.g., from the cancer of the individual) comprises cancer cells that express HER2 on their cell surface at a level of IHC3+, as measured by IHC assay (IHC3+). In some embodiments, a sample obtained from the individual (e.g., from the cancer of the individual) comprises cancer cells that express HER2 on their cell surface at a level of IHC2+, as measured by IHC assay (IHC2+); and a sample obtained from the individual (e.g., from the cancer of the individual) comprises cancer cells that exhibit HER2 gene amplification, as measured by ISH assay (IHC2+/ISH+). In some embodiments, a sample obtained from the individual (e.g., from the cancer of the individual) comprises cancer cells that express HER2 on their cell surface at a level of IHC2+, as measured by IHC assay (IHC2+); and a sample obtained from the individual (e.g., from the cancer of the individual) comprises cancer cells that exhibit HER2 gene amplification, as measured by FISH assay (IHC2+/FISH+). In some embodiments, a sample obtained from the individual (e.g., from the cancer of the individual) is IHC3+. In some embodiments, a sample obtained from the individual (e.g., from the cancer of the individual) is IHC3+/ISH+. In some embodiments, a sample obtained from the individual (e.g., from the cancer of the individual) is IHC3+/FISH+.

In some embodiments, the cervical cancer is squamous cell carcinoma. In some embodiments, the cervical cancer is adenocarcinoma. In some embodiments, the cancer is recurrent or metastatic cervical cancer. In some embodiments, the cancer has a primary FIGO stage of IIB or above.

In some embodiments, the cervical cancer is recurrent or metastatic cervical cancer. In some embodiments, the cervical cancer has been previous treated. In a further embodiment, the cervical cancer has been previously treated with at least one of platinum-containing standard therapy. In a further embodiment, the individual received at least four cycles of an adjuvant platinum-containing regimen, and progressed or recurred within 6 months from the end of at least four cycles. In some embodiments, administration of the antibody-drug conjugate and anti-PD-1 antibody is second-line therapy, third-line therapy, or fourth-line therapy. In some embodiments, the cells of the cervical cancer express HER2. In a further embodiment, a sample obtained from the individual comprises cancer cells that express HER2 on their cell surface at a level of IHC 1+, IHC 2+, or IHC 3+. In a further embodiment, the sample obtained from the individual comprises cancer cells that express HER2 on their cell surface at a level of IHC 2+ or IHC 3+, and are positive for HER2 expression by fluorescence in situ hybridization (FISH+). In some embodiments, a sample obtained from the individual comprises cancer cells that express PD-L1. PD-L1 refers to the 40 kDa transmembrane protein Programmed death-ligand 1, also known as cluster of differentiation 274 (CD274) or B7 homolog (B7-H1). Without being bound by theory, the binding of PD-L1 to the inhibitory checkpoint molecule PD-1 results in a reduction in proliferation of antigen-specific T-cells in lymph nodes.

### Individuals to be Treated

In some embodiments, e.g., prior to treatment according to the methods disclosed herein, the individual has been previously treated for the cancer. In some embodiments, the individual has recurrent or metastatic cervical cancer. In some embodiments, the cancer has previously been treated. In some embodiments, the cancer has been previously treated with at least first-line platinum-based therapy. In some embodiments, the cancer has been previously treated with radiotherapy. In some embodiments, the administration of the antibody-drug conjugate according to the methods of the present disclosure is a second-line therapy, third-line therapy, fourth-line therapy, or post-fourth-line therapy. In some embodiments, the individual is not a candidate for surgery or radiotherapy.

In some embodiments, e.g., prior to treatment according to the methods disclosed herein, the individual is at least 18 years of age. In some embodiments, e.g., prior to treatment according to the methods disclosed herein, the individual is at least 18 years of age or considered an adult in the jurisdiction in which the study is taking place at the time of consent. In some embodiments, the individual has an expected survival period of greater than or equal to 12 weeks before treatment. In some embodiments, e.g., prior to treatment according to the methods disclosed herein, the individual has measurable disease according to RECIST v1.1 guidelines. In some embodiments, e.g., prior to treatment according to the methods disclosed herein, the individual has measurable lesions according to RECIST v1.1 guidelines.

In some embodiments, e.g., prior to treatment according to the methods disclosed herein, the individual has disease (e.g., cervical cancer) demonstrating HER2 expression 1+, 2+, or 3+, as determined by local IHC testing on a fresh or archival tumor tissue (IHC1+, IHC2+, IHC3+, respectively). In some embodiments, e.g., prior to treatment according to the methods disclosed herein, the individual has disease (e.g., cervical cancer) demonstrating PD-L1 expression, as determined by in fresh or archival tumor tissue.

In some embodiments, e.g., prior to treatment according to the methods disclosed herein, the individual has an Eastern Cooperative Oncology Group (ECOG) Performance Status score of 0 or 1. In some embodiments, e.g., prior to treatment according to the methods disclosed herein, the individual has one, some, or all of the following lab values: Left ventricular ejection fraction (LVEF)≥50%; Hemoglobin≥9 g/dL; Absolute neutrophil count (ANC)≥1.5×10⁹/L; Platelets≥100×10⁹/L; Serum total bilirubin≤1.5 times the upper limit of normal (ULN); ALT and AST≤2.5×ULN when there is no liver metastasis, and ALT and AST≤5×ULN when there is liver metastasis; Serum creatinine≤1.5×ULN or creatinine clearance rate (CrCl)≥50 mL/min according to Cockcroft-Gault formula method.

In some embodiments, the individual is not suitable for surgery or radiotherapy. In some embodiments, the individual is a human.

### Administration

The concentration of a protein-based drug substance can be estimated using various methods that typically reference a comparative standard. Methods and reference standards evolve over time to be more precise and accurate in determining protein concentrations. In some embodiments, the dose is measured using the BSA-based Extinction Coefficient (EC) method. The BSA-based Extinction Coefficient (EC) method is a UV spectrophotometric method for determining the protein concentration using the sample's absorbance and an extinction coefficient (EC). Exemplary protocols are provided in Example 2 and Example 4; briefly, the absorbance of the sample at 280 nm with scatter-correction at 320 nm is read over multiple pathlengths, producing a slope (Abs/mm) which is then used to calculate protein concentration using the Beer-Lambert law and a product-specific extinction coefficient of 1.07 (mg/ml⁻¹)·cm⁻¹. In some embodiments, the antibody-drug conjugate is administered to the individual at a dose of 2.0 mg/kg, measured using the BSA-based EC method. In other embodiments, the dose contains the same mole-quantity of the drug as 2.0 mg/kg measured using the BSA-based EC method, but is measured using another method and therefore expressed differently, for example, as 1.5 mg/kg dose as measured using a disitamab vedotin-based EC method. Briefly, the absorbance of the sample at 280 nm with scatter-correction at 320 nm is read over multiple pathlengths, producing a slope (Abs/mm) which is then used to calculate protein concentration using the Beer-Lambert law and a different extinction coefficient.

### Administration of Anti-HER2 Antibody-Drug Conjugate Monotherapy

In some embodiments, the antibody-drug conjugate is administered to the individual at a dose of about 2.0 mg/kg. In some embodiments, the antibody-drug conjugate is administered to the individual at a dose of about 2.0 mg/kg, wherein the dose is measured using the BSA-based method. In some embodiments, the antibody-drug conjugate is administered to the individual at a dose of 2.0 mg/kg. In some embodiments, the antibody-drug conjugate is administered to the individual at a dose of 2.0 mg/kg, wherein the dose is measured using the BSA-based method. In some embodiments, the antibody-drug conjugate is administered intravenously to the individual.

In some embodiments, the antibody-drug conjugate is administered to the individual every 2 weeks or every 14 days (Q2W). In some embodiments, the antibody-drug conjugate is disitamab vedotin, and the method comprises intravenously administering disitamab vedotin to the individual at 2.0 mg/kg every two weeks or every 14 days (Q2W). In some embodiments, the antibody-drug conjugate is administered to the individual every 11 to 17 days. In some embodiments, the antibody-drug conjugate is administered to the individual every 11 days. In some embodiments, the antibody-drug conjugate is administered to the individual every 12 days. In some embodiments, the antibody-drug conjugate is administered to the individual every 13 days. In some embodiments, the antibody-drug conjugate is administered to the individual every 14 days. In some embodiments, the antibody-drug conjugate is administered to the individual every 15 days. In some embodiments, the antibody-drug conjugate is administered to the individual every 16 days. In some embodiments, the antibody-drug conjugate is administered to the individual every 17 days. In some embodiments, the antibody-drug conjugate is disitamab vedotin, and the method comprises intravenously administering disitamab vedotin to the individual at 2.0 mg/kg every 11 to 17 days.

In some embodiments, the administration of the antibody-drug conjugate according to the methods of the present disclosure is a second-line therapy. In some embodiments, the administration of the antibody-drug conjugate according to the methods of the present disclosure is a third-line therapy. In some embodiments, the administration of the antibody-drug conjugate according to the methods of the present disclosure is a fourth-line therapy. In some embodiments, the administration of the antibody-drug conjugate according to the methods of the present disclosure is post-fourth-line therapy.

In some embodiments, administration of the antibody-drug conjugate results in a complete response (CR) or partial response (PR) in the individual. In some embodiments, the time to onset of effect is less than two months. In some embodiments, the time to onset of effect is less than or equal to 1.5 months. In some embodiments, the duration of response is longer than five months. In some embodiments, the duration of progression free survival is longer than four months. In some embodiments, response to treatment according to the methods disclosed herein is evaluated using RECIST v1.1 criteria. Exemplary criteria are provided below.

Response Evaluation Criteria for Solid Tumors (RECIST) Version 1.1
(Eisenhauer, E. A. et al. (2009) Eur J Cancer 45(2): 228-247)

| Term | Definition |
|---|---|
| Complete Response (CR) | Disappearance of all target lesions. Any pathological lymph nodes (whether |
| | target or non-target) must have reduction in short axis to <10 mm |
| Partial Response (PR) | A ≥30% decrease in the sum of diameters of target lesions (longest for non-nodal |
| | target lesions and the short axes for nodal target lesions), taking as reference the |
| | baseline sum diameters |
| Stable Disease (SD) | Neither sufficient shrinkage to qualify for PR nor sufficient increase to qualify for |
| | PD, taking as reference the smallest sum diameters while on study |
| Progressive Disease (PD) | A ≥20% relative increase in the sum of diameters of target lesions, taking as |
| | reference the smallest sum on study (including baseline sum if that is the smallest |
| | on study). In addition, the sum must also demonstrate an absolute increase ≥5 mm |
| | The appearance of 1 or more new lesions is also considered progression |

In some embodiments, the individual is a human.

Administration of Anti-HER2 Antibody Drug Conjugate in Combination with Zimberelimab

In some embodiments, the antibody-drug conjugate is administered to the individual at a dose of about 1.5-2.0 mg/kg. In some embodiments, the antibody-drug conjugate is administered to the individual at a dose of about 1.5 mg/kg. In some embodiments, the antibody-drug conjugate is administered to the individual at a dose of about 1.5 mg/kg, wherein the dose is measured using the universal protein concentration assay method. In some embodiments, the antibody-drug conjugate is administered to the individual at a dose of 1.5 mg/kg. In some embodiments, the antibody-drug conjugate is administered to the individual at a dose of 1.5 mg/kg, wherein the dose is measured using the universal protein concentration assay method. In some embodiments, the antibody-drug conjugate is administered intravenously to the individual. In some embodiments, the antibody-drug conjugate is administered to the individual at a dose of about 2.0 mg/kg. In some embodiments, the antibody-drug conjugate is administered to the individual at a dose of about 2.0 mg/kg, wherein the dose is measured using the universal protein concentration assay method. In some embodiments, the antibody-drug conjugate is administered to the individual at a dose of 2.0 mg/kg. In some embodiments, the antibody-drug conjugate is administered to the individual at a dose of 2.0 mg/kg, wherein the dose is measured using the universal protein concentration assay method. In some embodiments, the dose is measured using the universal protein concentration assay method. In some embodiments, the antibody-drug conjugate is administered to the patient every two weeks or every 14 days. In some embodiments, the antibody-drug conjugate is administered to the individual at a dose of 2.0 mg/kg every two weeks or every 14 days. In some embodiments, the antibody-drug conjugate is administered to the individual at a dose of 1.5 mg/kg every two weeks or every 14 days. In some embodiments, the antibody-drug conjugate is administered intravenously to the individual. In some embodiments, the anti-PD-1 antibody is administered to the individual at a dose of about 240 mg. In a further embodiment, the anti-PD-1 antibody is administered to the individual at a dose of 240 mg. In some embodiments, the anti-PD-1 antibody is administered to the individual every two weeks or every 14 days. In some embodiments, the antibody-drug conjugate is disitamab vedotin administered to the individual at a dose of 2.0 mg/kg once every two weeks or 14 days, and the anti-PD-1 antibody is zimberelimab administered to the individual at a dose of 240 mg once every two weeks or 14 days. In some embodiments, the antibody-drug conjugate is disitamab vedotin administered to the individual at a dose of 1.5 mg/kg once every two weeks or 14 days, and the anti-PD-1 antibody is zimberelimab administered to the individual at a dose of 240 mg once every two weeks or 14 days. In some embodiments, administration of the antibody-drug conjugate and administration of the anti-PD-1 antibody to the individual are sequential. In a further embodiment, administration of the antibody-drug conjugate to the individual occurs prior to administration of the anti-PD-1 antibody to the individual. In another embodiment, administration of the antibody-drug conjugate to the individual occurs at least 30 minutes prior to administration of the anti-PD-1 antibody to the individual. In some embodiments, the antibody-drug conjugate and the anti-PD-1 antibody are administered to the individual every two weeks or 14 days for at least 6 weeks±7 days. In some embodiments, the antibody-drug conjugate and the anti-PD-1 antibody are administered to the individual every two weeks or 14 days for a maximum of 24 months. In some embodiments, the antibody-drug conjugate is administered to the individual every 11 to 17 days. In some embodiments, the antibody-drug conjugate is administered to the individual every 11 days. In some embodiments, the antibody-drug conjugate is administered to the individual every 12 days. In some embodiments, the antibody-drug conjugate is administered to the individual every 13 days. In some embodiments, the antibody-drug conjugate is administered to the individual every 14 days. In some embodiments, the antibody-drug conjugate is administered to the individual every 15 days. In some embodiments, the antibody-drug conjugate is administered to the individual every 16 days. In some embodiments, the antibody-drug conjugate is administered to the individual every 17 days. In some embodiments, the antibody-drug conjugate is disitamab vedotin, and the method comprises intravenously administering disitamab vedotin to the individual at 2.0 mg/kg every 11 to 17 days.

In some embodiments, administration of the antibody-drug conjugate and anti-PD-1 antibody results in a complete response (CR) or partial response (PR) in the individual. In some embodiments of the above aspects, which may be combined with any of the previous embodiments, administration of the antibody-drug conjugate and anti-PD-1 antibody results in an objective response rate (ORR) greater than 15%. In some embodiments of the above aspects, which may be combined with any of the previous embodiments, administration of the antibody-drug conjugate and anti-PD-1 antibody results in an objective response rate (ORR) greater than 25%.

In some embodiments, the administration of the antibody-drug conjugate according to the methods of the present disclosure is a second-line therapy. In some embodiments, the administration of the antibody-drug conjugate according to the methods of the present disclosure is a third-line therapy. In some embodiments, the administration of the antibody-drug conjugate according to the methods of the present disclosure is a fourth-line therapy. In some embodiments, the administration of the antibody-drug conjugate according to the methods of the present disclosure is post-fourth-line therapy.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Phase 2 Open-Label Basket Study of Disitamab Vedotin Monotherapy in Adult Subjects with HER2-Expressing (HER2-Positive and HER2 Low-Expression) Cervical Cancer

Overall survival (OS) and prognosis for patients with metastatic/recurrent cervical cancer are poor. There is an urgent need for novel therapies for metastatic/recurrent cervical cancer that can provide clinical benefit.

### Design

This example describes an open-label, multi-center Phase 2 basket design clinical study designed to assess the efficacy and safety of disitamab vedotin monotherapy for the treatment of subjects with recurrent or metastatic cervical cancer with HER2 expression defined by IHC level 1+, 2+, and 3+ who had previously received at least first-line platinum-based standard therapy.

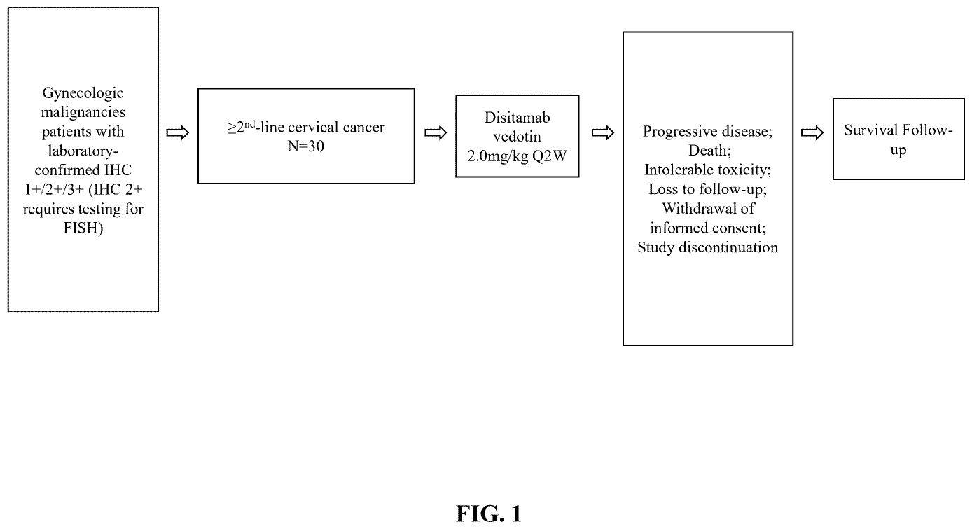

A schema for the study is shown in FIG. 1.

The cervical cancer cohort enrolled 30 subjects. At least five subjects had cervical cancer defined by IHC level 1+, at least five subjects had cervical cancer defined by IHC level 2+, and at least five subjects had cervical cancer defined by IHC level 3+. There were no requirements for IHC level for the cancers of the remaining 15 subjects. HER2 expression included HER2 positive and HER2 low expression. HER2 positive was defined by the immunohistochemical IHC 2+/fluorescence in situ hybridization technique+ (FISH+), or IHC 3+ judged by the central laboratory. HER2 low expression was defined as IHC 2+/FISH- or IHC+ as determined by the central laboratory. The Molecular Pathology Department, Cancer Hospital, Chinese Academy of Medical Sciences, served as the laboratory of this study site to confirm HER2 status (including IHC and FISH tests).

Based on previous study data, the ORR (evaluated by IRC) of subjects was estimated to be about 30%. In the assuming of the estimated ORR, the effectiveness evaluation of 30 subjects was conducted right after the tumor assessment was completed, the confidence interval (CI) for the ORR (evaluated by IRC) was expected to be around 30%+/−18% (95% Clopper-Pearson CI).

In general, subjects must have had recurrent or metastatic cervical cancer, had failed at least first-line platinum-based standard therapy, and had at least 1+ HER2 immunohistochemistry (see section on specific inclusion criteria). Subjects must have had measurable disease, Eastern Cooperative Oncology Group (ECOG) performance status 0 to 1, and adequate baseline hepatic, renal, and hematologic function. Subjects must not have received prior ADC therapy, or have had clinically significant cardiopulmonary disease, systemic diseases that have not been stably controlled by researchers, active autoimmune diseases requiring systemic treatment, or central nervous system (CNS) metastases and/or cancerous meningitis (see section on specific exclusion criteria).

Eligible subjects were treated with disitamab vedotin at a dose of 2.0 mg/kg (dose calculated using a bovine-serum albumin-based (BSA-based) extinction coefficient method) every 2 weeks (dosing window is from one day ahead to two days later in all cycles) by intravenous infusion. Subjects in this study must have had evaluable lesions per RECIST 1.1 criteria, and tumor assessments were performed every 6 weeks (+7 days) during the study until disease progression, death, intolerable toxicity, loss to follow-up, withdrawal of informed consent, or study termination. Tumor assessment timepoints were not affected by dose modification or interruption. An Independent Review Committee (IRC) was established in this study to review the subjects' tumor assessments, and the primary endpoint analysis was conducted at the time the subject completed the tumor assessment.

The safety of the study drug was evaluated during the study, and adverse events and concomitant medications were collected. The quality of life of the subjects was evaluated using the cervical cancer: FACT-Cx scale.

After the end of treatment (including withdrawal visit of treatment termination and the safety visit), all subjects were followed for survival every 3 months until death, loss to follow-up, withdrawal of informed consent, initiation of new anti-tumor therapy, or study termination at the discretion of the sponsor.

### Treatment

Eligible subjects received disitamab vedotin monotherapy treatment after enrollment, at a dose of 2.0 mg/kg, once every 2 weeks (the dosing time window in all cycles was-1 to 2 days), and the administration method was intravenous drip, until the disease progressed or toxicity became intolerable.

Objective tumor response was evaluated according to Response Evaluation in Solid Tumors version 1.1 (RECIST v1.1) criteria.

### Objectives and Endpoints

In this study, the primary objective was to evaluate the efficacy of disitamab vedotin monotherapy in subjects with previously treated recurrent or metastatic HER2 expressing (HER2-positive and HER2 low expression) cervical cancer. The endpoint for this object was objective response rate (ORR), defined as the percentage of participants with a complete response (CR) or partial response (PR) based on the Response Evaluation in Solid Tumors version 1.1 (RECIST v1.1) as assessed by the Independent Review Committee (IRC).

A secondary objective was to evaluate the safety of disitamab vedotin in subjects with previously treated recurrent or metastatic HER2 expressing (HER2-positive and HER2 low expression) cervical cancer. Endpoints included:
Objective Response Rate (ORR), defined as the percentage of participants with a complete response (CR) or partial response (PR) as assessed by the investigator.
Duration of response (DOR), defined as the time from the first documented objective response (CR or PR) to the first documented disease progression or death
Disease control rate (DCR)
Progression free survival (PFS), defined as the time from the first administration of study treatment to the first occurrence of disease progression or death from any cause, whichever comes first
Overall survival (OS), defined as the time from the first administration of study treatment to death from any cause
Adverse events (AEs)
Physical examination, vital signs, laboratory examinations, electrocardiogram, echocardiography
Cervical cancer: score of each domain and change from baseline in total score in FACT-Cx.

Another secondary objective was to evaluate the quality of life of disitamab vedotin in subjects with previously treated recurrent or metastatic HER2 expressing (HER2-positive and HER2 low expression) cervical cancer.

### Inclusion/Exclusion Criteria

Subjects were eligible to be included in the study only if all the following criteria were met:
1. Patients with histologically confirmed recurrent or metastatic cervical cancer who had failed at least one line of standard platinum-containing therapy or who had failed concurrent chemoradiotherapy; and Ineligible for surgery or radiotherapy;
2. Voluntarily agreed to participate in the research and sign an informed consent form;
3. Female, age ≥18 years old;
4. Expected survival period ≥12 weeks;
5. HER2 expression confirmed by the central laboratory: IHC 1+, 2+ or 3+; Subjects were able to provide samples of the primary or metastatic tumor site for HER2 testing (either paraffin blocks, paraffin-embedded sections, or fresh tissue sections were acceptable); Subjects with IHC2+ needed to be tested by FISH assay. Note: scoring criteria for HER2 testing was determined by the central laboratory;
6. With measurable lesions specified by RECIST 1.1 standard;
7. ECOG performance status 0 or 1 point;
8. Adequate organ functions that met the following criteria at screening (based on normal reference ranges of the study site):
   Left ventricular ejection fraction ≥50%;
   Hemoglobin ≥9 g/dL;
   Absolute neutrophil count (ANC) ≥1.5×10⁹/L;
   Platelets count ≥100×10⁹/L;
   Serum total bilirubin≤1.5× the upper limit of normal (ULN);
   Without liver metastasis: ALT and AST≤2.5×ULN, with liver metastasis: ALT and AST≤5×ULN;
   Blood creatinine≤1.5×ULN or creatinine clearance (CrCl) calculated according to the Cockcroft-Gault formula ≥50 mL/min;
9. Female subjects were surgically sterilized, post-menopausal, or agreed to use at least one medically approved contraceptive method (e.g., intrauterine contraceptive devices, contraceptives, or condoms) during study treatment and for 6 months after the end of the study treatment period, and must have had a negative blood pregnancy test within 7 days prior to study entry, and must be non-lactating.
10. Willing and able to comply with trial and follow-up procedures.

Exclusion criteria included:
1. Central nervous system metastasis and/or carcinomatous meningitis and subjects who had undergone treatment for brain metastases may have been eligible, provided that the subject had been in stable condition for at least 3 months, no radiographic evidence of disease progression within 4 weeks prior to the first dose, all neurological symptoms had recovered to baseline levels, there was no new or growth of brain metastasis, and the subject had discontinued radiation, surgery, or steroid therapy at least 28 days prior to the first dose. This exception did not include subjects with carcinomatous meningitis, who were not eligible even if clinically stable;
2. Had received anti-tumor treatment (including chemotherapy, radiotherapy, and immunotherapy) or participated in other clinical research treatments within 4 weeks before the start of study;
3. Toxicity from a previous anti-tumor treatments that had not been restored to CTCAE (version 5.0) Grade 0-1 (except for Grade 2 alopecia);
4. History of major surgery that had not fully recovered within 4 weeks prior to dose administration;
5. Pleural effusion and ascites with symptoms or requiring symptomatic treatment;
6. Serum virology (based on normal reference ranges of the clinical trial site):
   Subjects with positive HBsAg and positive HBV DNA copy number;
   Positive HCVAb (only subjects who had a negative HCV RNA in PCR test were eligible);
   Positive HIVAb;
7. Received live vaccine within 4 weeks prior to dose administration or planned to receive any vaccine during the study (except for vaccination against COVID-19);
8. New York Heart Association (NYHA) Grade 3 and more severe heart failure;
9. Severe arterial/venous thrombotic events or cardiovascular and cerebrovascular accidents within 1 year prior to dose administration, such as deep venous thrombosis (excluding asymptomatic intermuscular venous thrombosis not requiring special treatment), pulmonary embolism, cerebral infarction, cerebral hemorrhage, myocardial infarction, etc., except for asymptomatic lacunar infarction not requiring clinical intervention;
10. Active or progressive infection requiring systemic treatment, such as active tuberculosis;
11. Subjects who had uncontrolled systemic diseases, including diabetes mellitus, hypertension, cirrhosis, pneumonia, obstructive pulmonary disease, etc., as judged by the investigator;
12. Active autoimmune diseases requiring systemic treatment (such as the use of disease-modifying drugs, corticosteroids or immunosuppressive drugs) within 2 years prior to dose administration, and replacement therapies (e.g., thyroxine, insulin, physiological replacement of glucocorticoids due to renal or pituitary deficiency) are allowed;
13. History of other malignant tumors within 5 years prior to dose administration, except for
   Malignancies that could be cured after treatment (including but not limited to adequately treated thyroid cancer, cervical carcinoma in situ, basal or squamous cell skin cancer, or radical treatment of ductal carcinoma in situ of the breast);
14. Received allogeneic hematopoietic stem cell transplantation;
15. Received treatment with other antibody-conjugated drugs;
16. Known hypersensitivity to recombinant humanized anti-HER2 monoclonal antibody-MMAE conjugate or any of its components;
17. Any other disease, metabolic disorders, or abnormal findings upon physical examinations or laboratory examinations that made the subject unsuitable for receiving the investigational drug, would affect the interpretation of study outcomes, or posed risks to patient safety, as determined by the investigator;
18. Subject was assessed to have insufficient adherence to this clinical study.

### Concomitant Medication and Concomitant Therapy

Permitted concomitant medication were as follows. During the study, the investigator was allowed to provide appropriate drug/non-drug symptomatic or supportive care according to the disease condition of the subject. All concomitant medication/treatment was recorded, including prescription drugs, over-the-counter drugs, etc. If there were changes in dose, frequency, etc. during concomitant medication/treatment, the changes were recorded. During the study, non-targeted lesions could be treated locally (including local radiotherapy, biopsy, surgical resection and puncture and drainage of third space effusion) if judged necessary by the investigator.

Prohibited concomitant therapy were as follows. The following treatments were prohibited for subjects during the screening and treatment period:
Surgical treatment for the tumor diseases (except for surgical resection of non-target lesions; see permitted concomitant medication);
Biological therapy for tumor diseases (including immunotherapy, targeted therapy and cell therapy, etc.);
Radiotherapy for tumor diseases (except for local radiotherapy of non-target lesions, see permitted concomitant medication);
Endocrine therapy for tumor diseases;
Other treatments (such as treatment with anti-tumor Traditional Chinese Medicine [Chinese patent medicine], etc.), except the treatments for the tumor diseases specified in this protocol;
Other non-marketed investigational drugs except disitamab vedotin;
Received live vaccine within 28 days prior to dose administration or planned on receiving any vaccine during the study (except for vaccination against COVID-19);
Other prohibited medications/treatments specified in the exclusion criteria for this study;
Potent inhibitors or inducers of cytochrome P450 (CYP3A4):
   Potent inhibitors of cytochrome P450 (CYP3A) include aprepitant, idelalisib, boceprevir, posaconazole, troleandomycin, diltiazem, danapril and ritonavir, elvitegravir and ritonavir, elvitegravir and ritonavir, partiaprevir and ritonavir, ombitavir and dasabuvir, saquinavir and ritonavir, tipranavir and ritonavir, voriconazole, fosaprepitant, fluconazole, floxamide, erythromycin, cyclosporin, ciprofloxacin, dronedarone, cobicistat, crizotinib, conivaptan, clarithromycin, clotrimazole, lopinavir and ritonavir, ritonavir, chlorzoxazone, lomitamide, ranitidine, ranolazine, nefazodone, nelfinavir, grapefruit juice, ketoconazole, telaprevir, tofisopam, tacrolimus, ticagrelor, verapami, cimetidine, itraconazole, imatinib, isostatin, ivatarin, ivastatone.
   Potent inducers of cytochrome P450 (CYP3A) include: armodafinil, phenylurea, phenytoin, bosentan, carbamazepine, rifampicin, rufinamide, mitotane, modafinil, St. John's wort, efavirenz, etravirine;
After the subjects completed the treatment with the investigational drugs, their treatments were performed by the investigator according to the clinical routine and were no longer limited by the above requirements.

### Statistical Analysis

All statistical tests were two-sided with a significance level of 5%, using 95% CIs, unless otherwise specified. Descriptive statistics for continuous variables included the number of subjects, mean, standard deviation, median, minimum, and maximum. Categorical variables were used for descriptive statistics using frequencies and percentages.

Safety: All patients enrolled in the trial and received at least one dose of the study drug were included in the safety analysis set. A table of overall frequency of occurrence, reporting of adverse events by severity, and by drug-related classification was provided. Laboratory variables and vital signs were reported with descriptive statistics. All adverse events and abnormal laboratory values were evaluated according to NCI-CTCAE v5.0.

Efficacy: All the enrolled patients with cervical cancer were included in the ITT Analysis Set. Objective tumor response was evaluated according to RECIST version 1.1 criteria. Descriptive results of tumor evaluation was presented by cohort and IHC expression level. The number and proportion of best response assessments (CR, PR, SD, PD and NE) was summarized. The number and proportion of subjects with ORR and DCR was calculated and 95% CI was provided. For PFS, DOR, OS, the median, first quartile, third quartile, and their 95% two-sided confidence intervals was calculated using the Kaplan-Meier method and survival curves were plotted.

### Example 2: Bovine Serum Albumin (BSA)-Based Extinction Coefficient (EC) Method

This example describes a method used to determine the concentration of disitamab vedotin using a bovine serum albumin-based (BSA-based) UV spectrophotometric method that determines the protein concentration using the absorbance of the sample and an extinction coefficient (EC).

The Lowry colorimetric method was previously used to determine disitamab vedotin concentration using BSA as the reference standard. Alternatively, UV spectrophotometric methods were also previously developed to determine the protein concentration using the absorbance of the sample and an extinction coefficient. The BSA-based extinction coefficient method used in the present application uses an EC value that was established mathematically to align the Lowry colorimetric and UV spectrophotometric methods, such that both yield the same estimated measurements of disitamab vedotin protein concentration.

Briefly, the sample is transferred to a vessel, and the absorbance at 280 nm with scatter-correction at 320 nm is read over multiple pathlengths using a SoloVPE system. This produces a slope (Abs/mm) which is then used to calculate protein concentration using the Beer-Lambert law and a product-specific extinction coefficient.

Calculations and Data Analysis:
The concentration will be calculated by the SoloVPE software, and results can be included in a report generated by the software. Alternatively, calculate the protein concentration using the following equation:

$$Concentration = \frac{{m}_{280}}{ε} \times \frac{10 mm}{1 cm}$$

$$Where : {m}_{280} = slope at 280 nm ( {mm}^{- 1} )$$

$$ε = extinction coefficient$$

Calculate the average concentration from the duplicate or triplicate measurements to three decimal places.
For lyophilized samples: Calculate drug product content for lyophilized product vials using the following equation:

$$Drug product content = Concentration ( mg / mL ) \times Final Reconstitution Volume ( mL / vial )$$

### Example 3: a Single-Arm, Multicenter, Open-Label Phase II Clinical Study Evaluating the Efficacy and Safety of Disitamab Vedotin for Injection in the Monotherapy Treatment of HER2 Expressing and Platinum-Based Recurrent Cervical Cancer

Disitamab vedotin for injection is an ADC drug that targets HER2. A Phase II, open-label, multicenter basket design study (NCT04965519) of the efficacy and safety of disitamab vedotin (also known as RC48) monotherapy in the treatment of HER2 expressing gynecologic malignancies, performed as in Example 1 above, is ongoing.

Methods: The cervical cancer cohort included patients with recurrent or metastatic cervical cancer who had failed at least first-line of platinum-based standard therapy and had at least 1+ HER2 immunohistochemistry. The treatment regimen was disitamab vedotin intravenous injection in a monotherapy treatment, 2 mg/kg Q2W, until the disease progressed or toxicity became intolerable. The primary endpoint was IRC-evaluated objective response rate (ORR) based on RECIST1.1, and secondary endpoints included investigator-evaluated ORR, duration of response (DoR), disease control rate (DCR), progression-free survival (PFS), overall survival (OS), and safety.

Results: From Jan. 24, 2022 to Apr. 30, 2023, a total of 17 patients with cervical cancer were enrolled in the study and received at least one dose treatment, with a median age of 53 years (range: 35-66 years). The number of patients in line two therapy (i.e., treatment with disitamab vedotin was second-line therapy; i.e., the patient had previously only been treated with first-line therapy), line three therapy (i.e., treatment with disitamab vedotin was third-line therapy; i.e., the patient had previously been treated with first- and second-line therapies), and line four therapy (i.e., treatment with disitamab vedotin was fourth-line therapy; i.e., the patient had previously been treated with first-, second-, and third-line therapies) were 10 (58.8%), 5 (29.4%) and 2 (11.8%), respectively. The baseline ECOG score of most patients (58.8%, 10 patients) was 1; 58.8% (10/17) of patients had primary FIGO stage IIB or above. The baseline pathological type of 13 (76.5%) patients was squamous cell carcinoma, and the baselines pathological type of the remaining four patients (23.5%) was adenocarcinoma. 88.2% (15 patients) had received prior radiotherapy. The number of dosing for all patients ranged from 1 to 16 doses; the median dosing was 7.0. For the 16 patients who reached the effectiveness evaluation, the confirmed ORR was 43.8% (7/16) (95% CI: 19.8%, 70.1%), the median DoR was 5.45 months (95% CI: 2.10, NE), and the median time to onset of effect was 1.5 months. The DCR was 87.5% (14/16) (95% CI: 61.7%, 98.4%), the median PFS was 4.17 months (95% CI: 2.92, 6.90), and the OS data were not yet mature. Subgroup analysis showed that the ORRs for line two, line three, and line four patients (i.e., patients receiving disitamab vedotin as second-line, third-line, and fourth-line therapy, respectively) were 33.3% (3/9), 60% (3/5) and 50% (1/2), respectively. The ORRs of squamous cell carcinoma and adenocarcinoma was 50% (6/12) and 25% (1/4), respectively. Among all 17 patients with cervical cancer enrolled, the most common TRAE included white blood cell count decreased (52.9%), aspartate aminotransferase level increased (52.9%), alanine aminotransferase level increased (47.1%), anemia (47.1%), and Y-glutamyltransferase level increased (35.3%). Grade 3 TRAE included neutrophil count decreased (11.8%) and γ-glutamyltransferase level increased (11.8%). SAE occurred in 2 patients (11.8%), and no disitamab vedotin-related deaths occurred.

Conclusions: Disitamab vedotin has a controllable safety in patients with HER2-expressing recurrent or metastatic cervical cancer, showing positive efficacy, and has the potential to become a powerful new treatment for HER2-expressing cervical cancer patients who have recurred after multiple treatment lines. Study treatment is composed of disitamab vedotin 2.0 mg/kg Q2W (dose is calculated using a bovine serum albumin (BSA)-based extinction coefficient method) until disease progression or unacceptable toxicity.

### Example 4: Concentration Calculation of Disitamab Vedotin

This describes the method for a universal protein concentration assay, wherein the protein has the maximum UV absorption at 280 nm. The absorbance value of the sample at this wavelength is proportional to the protein concentration, which is calculated according to the Beer-Lambert law.

### Methods

Main equipment: UV-Vis spectrophotometer PE model: Lambda 365

Analysis process: The sample was diluted to 0.5±0.1 mg/ml with ultrapure water (the dilution multiple was determined according to the labeled sample concentration, and the dilution multiple≤10 each time), and the final volume of the sample after dilution was ≥1 ml. Two sample solutions were prepared in parallel, labeled A and B respectively, diluted and then measured the absorbance of A and B solutions at 260 nm, 280 nm, 320 nm, 325 nm, 330 nm, 335 nm, 340 nm, 345 nm, 350 nm, and each solution was measured twice in parallel.

### Data Analysis

Concentration Calculation: The formula for calculating protein concentration of the sample solution is listed below, and the average protein concentration of the two parallel solutions is used as the final value.

$$protein concentration ( mg / ml ) = \frac{{A}_{280}}{1.07} \times sample dilution multiple$$

$$Where : 1.07 = the extinction coefficient value ( {( mg / ml )}^{- 1} \cdot {cm}^{- 1} ) of disitamab vedotin ;$$

$${A}_{280} = the average absorbance of the sample solution at 280 nm$$

### System Suitability

1) The CV values of absorbance values were measured in parallel≤2.0%
2) The Blank absorbance value A₂₈₀≤0.001

### Standard Limit

The protein concentration of sample is 9.0˜11.0 mg/ml.

### Example 5: a Single-Arm, Open-Label, Multicenter Phase II Clinical Study of Disitamab Vedotin (DV) in Combination with Zimberelimab in Subjects with Recurrent or Metastatic Cervical Cancer with HER2-Expression Who have Failed at Least One Line of Platinum-Containing Standard Therapy

Overall survival (OS) and prognosis for patients with metastatic/recurrent cervical cancer are poor. There is an urgent need for novel therapies for metastatic/recurrent cervical cancer that can provide clinical benefit.

### Objectives

### Part I: Safety Run-In

Primary Objective: to evaluate the safety of DV in combination with zimberelimab in patients with HER2-expressing cervical cancer who have failed at least one line of platinum-containing standard therapy.

Secondary Objectives:
Combined evaluation with Simon's two-stage Phase I of the efficacy of DV in combination with zimberelimab in subjects with HER2-expressing cervical cancer who have failed at least one line of platinum-containing standard therapy;
Evaluation of quality of life of DV in combination with zimberelimab in patients with HER2-expressing cervical cancer who have failed at least one line of platinum-containing standard therapy;
Evaluation of the antitumor activity of DV in combination with zimberelimab in patients with HER2-expressing cervical cancer who have failed at least one line of platinum-containing standard therapy.

Exploratory Objective: to evaluate potential correlations between biomarkers and effectiveness.

### Part II: Dose Expansion Phase (Simon's Two-Stage)

Primary Objective: to evaluate the effectiveness of DV in combination with zimberelimab in patients with HER2-expressing cervical cancer who have failed at least one line of platinum-containing standard therapy.

Secondary Objectives:
To evaluate the safety of DV in combination with zimberelimab in patients with HER2-expressing cervical cancer who have failed at least one line of platinum-containing standard therapy;
To evaluate quality of life of DV in combination with zimberelimab in patients with HER2-expressing cervical cancer who have failed at least one line of platinum-containing standard therapy;
To evaluate the antitumor activity of DV in combination with zimberelimab in patients with HER2-expressing cervical cancer who have failed at least one line of platinum-containing standard therapy;
To evaluate the Simon's two-stage phase II pharmacokinetic (PK) profile of DV in combination with zimberelimab in patients with HER2-expressing cervical cancer who have failed at least one line of platinum-containing standard therapy;
To evaluate the Simon's two-stage phase II immunogenicity of DV in combination with zimberelimab in patients with HER2-expressing cervical cancer who have failed at least one line of platinum-containing standard therapy.

Exploratory Objective: to evaluate potential correlations between biomarkers and effectiveness.

### Design

The study is a single-arm, open-label, multicenter Phase II pivotal clinical study that plans to enroll 116 subjects with HER2-expressing recurrent or metastatic cervical cancer who have failed at least one line of platinum-containing standard therapy.

HER2 expression includes immunohistochemical IHC 1+, IHC2+, and IHC3+ as determined by the central laboratory, with IHC2+ and IHC3+ categorization requiring fluorescence in situ hybridization (FISH).

The clinical study is mainly divided into two parts:

### Part I: Safety Run-In

After signing an informed consent form, eligible subjects are enrolled into a 21-day dose-limiting toxicity (DLT) observation period. First disitamab vedotin (DV, also known as RC48-ADC) 2.0 mg/kg every two weeks or every 14 days (Q2W) is combined with zimberelimab injection 240 mg Q2W. In this period, dosing of DV is followed by zimberelimab injection after at least a 30 minute interval, every 2 weeks. One treatment cycle lasts six weeks. The combination dose is enrolling six subjects, and if ≤1 out of 6 patients experiences a dose-limiting toxicity (DLT), the study as a whole proceeds to the second part. If two or more of the six subjects experience DLTs, the dose will be adjusted downward to DV 1.5 mg/kg Q2W combined with zimberelimab 240 mg Q2W for DLT observation. The doses of DV are measured using the universal protein concentration assay, as in Example 4.

The first part of the Safety run-in phase DLT Observation Period is defined as an adverse reaction judged by the investigator or sponsor to be related to the trial drug (refer to NCI CTCAE version 5.0) on Day 21 of the first dose, and being associated with DV or combination therapy, but not with zimberelimab. See below for details:

(1) Hematological toxicity:
Grade 4 neutropenia >7 days (duration allows symptomatic supportive treatment);
Grade 4 anemia or thrombocytopenia or grade 3 thrombocytopenia with bleeding.
   (2) Non-hematological toxicity:
Grade ≥3 ALT/AST elevation with grade 2 or higher bilirubin elevation (excluding patients with baseline liver disease or liver metastases) without recovery to grade≤2 at 7 days or to grade≤1 at 14 days after adequate supportive treatment.
   (3) Subjects during the DLT evaluation period must meet the requirements of receiving at least 80% of the planned dose and the relevant examinations planned for the study trial. Non-completion of the condition is not evaluable for DLT (Subjects without DLT during the DLT observation period shouldn't participate in DLT analysis, but are included in other safety analysis).

Prophylactic medication is not allowed during DLT observation. However, after the use of the investigational drug, it is recommended to discuss with the sponsor in advance if support/symptomatic treatment is required in the judgment of the investigator (except for emergency situations such as endangering the life of the subject).

Based on the comprehensive consideration of the safety stage clinical data of the exploratory study of first part of safety run-in, further adjustment of Simon's two-stage dose regimen, dosage, dosing frequency, and dosing cycle is considered in the second part.

### Part II: Dose Expansion Phase (Simon's Two-Stage)

Step I: Enrollment of 38 subjects, of which 6-12 subjects will be used for the safety run-in, and the remaining will undergo Simon's two-stage Phase I. After completion of two tumor assessments, an ORR interim analysis is performed.

The first 38 subjects are analyzed for efficacy and all subjects who receive at least two treatment cycles are tested for futility. The adjustment of a is not considered, since only futility analysis of efficacy is performed. If the ORR for DV combined with zimberelimab is higher than 15%, the study continues to proceed.

Step II: Based on the results of Step 1, Step 2 is carried out in full. The enrollment deadline is based on the last subject who signs the protocol and gives informed consent. Final analysis of the ORR is performed after completing the enrollment of 78 subjects and conducting a 6-month follow-up after the enrollment of the last subject in this stage.

According to RECIST 1.1 criteria, subjects in this study must have evaluable lesions. Tumor evaluations are performed every 6 weeks (±7 days) during the study, and all patients continue treatment for a maximum of 24 months or until progressive disease, death, intolerable toxicity, loss to follow-up, withdrawal of knowledge, or study termination. Tumor evaluation time points are not affected by dose adjustments or interruptions.

Safety of the investigational drug of subjects is evaluated during the study period, while collecting adverse events and coadministration and evaluating the quality of life of the subjects using the Cervical Cancer: FACT-Cx Scale (every 6 weeks±7 days).

Exploratory index testing such as tumor tissue (fresh biopsy tissue or archived paraffin tissue available for analysis) is performed during the study (if feasible).

In the event of occurrence of intolerable toxicity with combination therapy, whether to withdraw from the clinical study or continue monotherapy with DV or zimberelimab until the disease progresses or toxicity is not tolerated can be determined by investigators.

After completion of treatment (including termination of treatment, exit visits and safety visits), all subjects are followed for survival every 3 months until death, loss to follow-up, withdrawal of knowledge, or termination of the study at the discretion of the sponsor.

### Study Population

Subjects with HER2 expression (IHC1+, IHC2+, and IHC3+) cervical cancer who have failed at least one line of platinum-containing standard therapy.

### Inclusion/Exclusion Criteria

Subjects are eligible to be included in the study only if all the following criteria are met:
1. a) Patients with histologically confirmed recurrent or metastatic HER2-expressing cervical cancer that has failed at least one line of standard platinum-containing therapy (patients are considered to have received one line of therapy if they have progressed or recurred while receiving an adjuvant platinum-containing regimen within 6 months of the end of at least 4 cycles); and
   b) Not suitable for surgery or radiotherapy;
2. Voluntarily participate and sign the informed consent form;
3. Female, ≥18 years old;
4. Expected survival ≥12 weeks;
5. The central laboratory confirmed HER2 expression: IHC 1+, 2+, or 3+; Subjects can provide HER2-detected primary or metastatic tumor specimens (paraffin blocks, paraffin-embedded sections, or fresh tissue sections); Subjects with IHC2+ will be tested with FISH. Note: The scoring criteria for HER2 tests are determined by the central laboratory;
6. Central laboratory confirmation of PD-L1 expression. Subjects must agree to provide tumor tissue samples sufficient to meet assay requirements for PD-L1 expression testing. This includes archived tumor samples (paraffin blocks or unstained sections in quantities that meet the testing requirements set by the Institute); if archived tumor tissue samples are not available, the subject agrees to receive re-biopsy of the tumor lesion. Note: Scoring criteria for PD-L1 testing are determined by the central laboratory;
7. Measurable lesions as defined by RECIST 1.1 criteria; measurable lesions located in the field of previous radiotherapy exposure or after localized treatment may be selected as target lesions if progression is confirmed;
8. ECOG physical status 0 or 1;
9. Adequate organ function should meet the following criteria during the screening period (normal values are based on the clinical trial center):
   left ventricular ejection fraction ≥50%;
   Blood routine (no blood components or growth factors are given within 14 days before the results are obtained): hemoglobin ≥9 g/dL; Absolute neutrophil count (ANC) ≥1.5×10⁹/L; Platelet ≥100×10⁹/L;
   Liver function: serum total bilirubin≤1.5 times the upper limit of normal (ULN); ALT and AST≤2.5×ULN without liver metastasis, ALT and AST≤5×ULN with liver metastasis;
   Renal function: serum creatinine≤1.5×ULN or creatinine clearance (CrCl) ≥50 mL/min according to Cockcroft-Gault formula method.
10. Female subjects should be surgically sterilized, postmenopausal, or agree to use at least one medically acceptable method of contraception (e.g., intrauterine device, contraceptives, or condoms) during study treatment and for 6 months after the end of the study treatment period, and must have a negative blood pregnancy test within 7 days prior to study entry, and must be non-lactating;
11. Willing and able to comply with trial and follow-up procedures.

Exclusion criteria include:
1. Central nervous system (CNS) metastases and/or carcinomatous meningitis and subjects who have undergone treatment for brain metastases may be eligible, provided that the subject has been in stable condition for at least 3 months, no radiographic evidence of disease progression within 4 weeks prior to the first dose, all neurological symptoms have returned to baseline levels, and no new or growth of brain metastasis, and the subject has discontinued radiation, surgery or steroid therapy at least 28 days prior to the first dose. This does not include subjects with carcinomatous meningitis, who are not eligible even if clinically stable;
2. Have received anti-tumor treatment (including chemotherapy, radiotherapy and immunotherapy) or participated in other clinical research treatments within 4 weeks before the start of study;
3. Toxicity from a previous anti-tumor treatment that has not recovered to CTCAE v5.0 Grade 0-1 (except for Grade 2 alopecia);
4. History of major surgery that has not fully recovered within 4 weeks prior to dose administration;
5. Serum virology (based on normal reference ranges of the clinical trial site):
   Subjects with positive HBsAg and positive HBV DNA copy number;
   Positive HCVAb (only subject who has a negative HCV RNA in PCR test will be eligible);
   Positive HIVAb.
6. Received live vaccine within 4 weeks prior to dose administration or plan on receiving any vaccine during the study (If need to be vaccinated against COVID-19, live or attenuated vaccines are not acceptable);
7. New York Heart Association (NYHA) Grade 3 or more severe heart failure;
8. A history of gastrointestinal perforation and/or fistula, intestinal obstruction (including incomplete intestinal obstruction requiring parenteral nutrition), inflammatory bowel disease or extensive enterectomy (partial colon resection or extensive intestinal resection with chronic diarrhea), Crohn's disease, ulcerative colitis, or chronic diarrhea within the past 6 months;
9. Serious arterial/venous thrombotic events or cardiovascular and cerebrovascular accidents within 1 year prior to dose administration, such as deep venous thrombosis (excluding asymptomatic intermuscular venous thrombosis not requiring special treatment), pulmonary embolism, cerebral infarction, cerebral hemorrhage, myocardial infarction, etc., except for asymptomatic lacunar infarction not requiring clinical intervention;
10. Active or advanced infection requiring systemic treatment, with severe infection within 4 weeks prior to first dosing, including but not limited to hospitalization for complications of infection, bacteremia, or severe pneumonia;
11. Active tuberculosis (Receiving anti-TB therapy or have received anti-TB therapy within 1 year prior to the first investigational drug);
12. Presence of systemic disease not under stable control as determined by the investigator, including diabetes mellitus, hypertension (systolic blood pressure ≥150 mmHg or diastolic blood pressure ≥100 mmHg after optimal medical therapy, hypertensive crisis or hypertensive encephalopathy history), and cirrhosis, etc.;
13. History of interstitial pneumonia, obstructive pulmonary disease, drug-induced pneumonia, radiation pneumonia, idiopathic pneumonia, or active pneumonia;
14. Clinically relevant hydronephrosis that cannot be alleviated by ureteral stents or percutaneous drainage;
15. Active autoimmune diseases requiring systemic therapy (such as the use of disease-modifying drugs, corticosteroids, or immunosuppressive drugs) within 2 years prior to dose administration (replacement therapies (e.g., thyroxine, insulin, or physiological replacement of glucocorticoids due to renal or pituitary deficiency) are allowed).
16. History of other malignant tumors within 5 years prior to dose administration, but except for.
   Malignancies that can be cured after treatment (including but not limited to adequately treated thyroid cancer, cervical carcinoma in situ, basal or squamous cell skin cancer, or radical treatment of ductal carcinoma in situ of the breast);
17. Received allogeneic hematopoietic stem cell transplantation;
18. Received treatment with other antibody-conjugated drugs;
19. Known to be allergic to DV and its components or to zimberelimab and other monoclonal antibodies;
20. Any other disease, metabolic disorder, or abnormal findings upon physical examination or laboratory examination that makes the subject unsuitable for receiving the investigational drug, affects the interpretation of study outcomes, or poses risks to patient safety, as determined by the investigator; or
21. Subject is assessed to have insufficient adherence to this clinical study.

### Independent Review Committee

The efficacy of tumor imaging assessment data of subjects is accessed per Independent Review Committee IRC) in a blinded manner (independent review among viewers).

### Evaluation Endpoints

### Part I: Safety Run-In

Primary Efficacy Endpoint:

### Safety Indicators:

Adverse events;
Vital signs, physical examination, laboratory examination, electrocardiogram, echocardiography

Secondary efficacy endpoints (combined evaluation with Simon's two-stage Phase I):
Objective response rate (ORR), as assessed by IRC;
Duration of response (DOR), as assessed by IRC;
Disease control rate (DCR), as assessed by IRC;
Progression-free survival (PFS), as evaluated by IRC;
Overall survival (OS), as evaluated by IRC

Quality of Life assessment Scale:
FACT-Cx scale scores for each domain, change from baseline in total score every 6 weeks±7 days

Exploratory evaluation indicators:
Potential association of biomarkers (HER2, PD-L1) with efficacy

### Part II: Dose Expansion Phase (Simon's Two-Stage)

Primary Efficacy Endpoint:
Objective response rate (ORR), as assessed by IRC

Secondary Efficacy Endpoints:
Objective response rate (ORR), as assessed by investigator;
Duration of response (DOR), as assessed by IRC;
Disease control rate (DCR), as assessed by IRC;
Progression-free survival (PFS), as evaluated by IRC;
Overall survival (OS), as evaluated by IRC.

Safety indicators:
Adverse events;
Vital signs, physical examination, laboratory examination, electrocardiogram, echocardiography.

Quality of Life assessment Scale:
FACT-Cx scale scores for each domain, change from baseline in total score every 6 weeks ±7 days.

Pharmacological indicators:
PK of serum DVs, total antibodies and free MMAE in subjects in Simon's two-stage Phase II;
Incidence, titer and neutralizing antibody (Nab) incidence of anti-DV antidrug antibodies (ADA) in Phase II of Simon's two-stage.

Exploratory evaluation indicators:
Potential association of biomarkers (HER2, PD-L1) with efficacy.

### Sample Size

### Part I: Safety Run-In

Part I is an exploratory study with a planned enrollment of 6-12 subjects. The sample size for this part is included in the Simon two-stage Phase I together with a combined analysis.

### Part II: Dose Expansion Phase (Simon's two-stage)

Part II uses a Simon optimal two-stage design to test the null hypothesis of ORR≤0.15 versus the alternative hypothesis ORR >0.15. The maximum sample size of the study is 116 cases (38 cases in Phase 1, with 6-12 subjects used for the safety access test first, and the remaining subjects for the Simon two-stage Phase I; and 78 cases in Phase 2), with an expected sample size of 64.61, and the probability of early termination is 0.659.

Sample size calculations are based on the assumptions that 1) DV in combination with zimberelimab would provide an ORR of 25%, and 2) the ORR of available treatments for advanced cervical cancer would be 15%. With a two-sided test level of α=0.05, the total sample size of 116 subjects would provide a test efficacy (certainty) of 80%.

Stage 1 of the study includes 38 participants and provides preliminary efficacy data to assess whether the preliminary efficacy data is sufficient to support the introduction of the product into Stage 2. If 6 or fewer subjects achieve objective response, the trial will be terminated early.

If the study proceeds to stage 2, 78 participants will continue to be enrolled, for a total of 116 participants in both phases. If the total number of people achieving objective response in both stages is less than or equal to 23, the investigational drug cannot be demonstrated to be effective.

### Concomitant Medication and Concomitant Therapy

### Permitted Concomitant Medication

During the study, the investigator is allowed to provide appropriate drug/non-drug symptomatic or supportive care according to the disease condition of the subject. All concomitant medication/treatment should be recorded, including prescription drugs, over-the-counter drugs, etc. If there are changes in dose, frequency, etc. during concomitant medication/treatment, the changes should be recorded.

In this study, the following concomitant treatments are permitted:
Drugs determined by the investigator to conform to protocol (e.g., for the treatment of disease-related symptoms and concomitant treatment of related AE).
Subjects who require long-term medication for underlying conditions such as hypertension and diabetes may continue to take medication.
During investigational therapy, local palliative radiotherapy is permitted for isolated lesions (non-target lesions) for symptom control and do not affect efficacy. Before proposed palliative radiotherapy, the investigator should inform the sponsor and start treatment after review by a medical monitor.
Allow supportive treatments that relieve tumor-related symptoms, such as bisphosphonate therapy for bone metastases.
Allow antiviral therapy as necessary and request antiviral therapy during the study if the subject is a hepatitis B virus carrier.
Allow temporary use of anti-allergy drugs, glucocorticoids, etc. for the treatment of allergic reactions. Temporary use of glucocorticoids is permitted for the treatment of breathing difficulties such as COPD or asthma.
Allow colony stimulating factors to be used according to guidelines.

### Prohibited Concomitant Therapy

The following treatments are prohibited for subjects during the screening and treatment period:
Surgical treatment for the tumor diseases (except for surgical resection of non-target lesions);
Biological therapy for tumor diseases (including immunotherapy, targeted therapy and cell therapy, etc.);
Patients should not receive immunomodulatory drugs such as thymosin, interleukin, interferon (except interleukin 11 for platelet elevation) that affect the efficacy of immunotherapy;
Radiotherapy for tumor diseases (except for local radiotherapy of non-target lesions);
Endocrine therapy for tumor diseases;
Except other treatments for the tumor diseases specified in this protocol (such as treatment with anti-tumor Traditional Chinese Medicine [Chinese patent drug]: Chinese patent drug with anti-tumor indications or Chinese herbal remedies for anti-tumor purposes should not be used in the course of this study);
Except DV and other investigational drugs for zimberelimab;
Have received a live vaccine within 4 weeks before the start of study dosing or planned to receive any vaccine (other than COVID-19 vaccine) during the study period. Live vaccines include, but are not limited to, measles, mumps, rubella, chickenpox, yellow fever, BCG, typhoid (oral) vaccines. Patients are allowed to receive inactivated virus vaccines for seasonal influenza; however, live attenuated flu vaccines are not allowed.
Corticosteroids, with the exception only of topical (e.g., topical skin application, inhaled, etc.) or systemic administration of medications at physiological doses (≤10 mg/day or equivalent doses of prednisone) for adverse events of underlying immune etiology, and other conditions specified in the protocol;
Other prohibited medications/treatments specified in the exclusion criteria for this study.
Potent inhibitors or inducers of cytochrome P450 (CYP3A₄):
   Potent inhibitors of cytochrome P450 (CYP3A) include aprepitant, idelalisib, boceprevir, posaconazole, troleandomycin, diltiazem, danapril and ritonavir, elvitegravir and ritonavir, elvitegravir and ritonavir, partiaprevir and ritonavir, ombitavir and dasabuvir, saquinavir and ritonavir, tipranavir and ritonavir, voriconazole, fosaprepitant, fluconazole, floxamide, erythromycin, cyclosporin, ciprofloxacin, dronedarone, cobicistat, crizotinib, conivaptan, clarithromycin, clotrimazole, lopinavir and ritonavir, ritonavir, chlorzoxazone, lomitamide, ranitidine, ranolazine, nefazodone, nelfinavir, grapefruit juice, ketoconazole, telaprevir, tofisopam, tacrolimus, ticagrelor, verapami, cimetidine, itraconazole, imatinib, isostatin, ivatarin, ivastatone.
   Potent inducers of cytochrome P450 (CYP3A) include: armodafinil, phenylurea, phenytoin, bosentan, carbamazepine, rifampicin, rufinamide, mitotane, modafinil, St. John's wort, efavirenz, etravirine.

After the subjects have completed the treatment with the investigational drugs, their treatments are performed by the investigator according to the clinical routine and will no longer be limited by the above requirements.

### Statistical Analysis

### Part I: Safety Run-In

Part I is the exploratory stage, and descriptive statistical analysis is performed according to the safety and other aspects of the treatment regimen. This part is analyzed for efficacy along with Simon's two-stage phase I.

### Part II: Dose Expansion Phase (Simon's Two-Stage)

For the primary efficacy endpoint of ORR assessed by IRC, the number and percentage of each group achieving an objective response ORR is calculated, as well as a precise bilateral 95% confidence interval (CI) based on the Clopper-Pearson method. The same method is used to analyze the ORR assessed by the investigator as well as the IRC, and DCR assessed by the investigator.

Time-event endpoints PFS, OS, DOR are calculated and the survival curve plotted using the Kaplan-Meier method. The associated median, first quartile, and third quartile will be calculated from the curves, and the two-sided 95% CI will be calculated using the Greenwood formula.

For the change from baseline in the quality-of-life endpoint FACT-Cx score, the least squares means, standard errors, and 95% bilateral confidence intervals are reported for each treatment group.

All statistical tests are two-sided with a significance level of 5%, using 95% CIs, unless otherwise specified. Descriptive statistics for continuous variables include the number of subjects, mean, standard deviation, median, minimum, and maximum. Categorical variables will be used for descriptive statistics using frequencies and percentages.

1. A method for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule;
wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6; and
wherein the cervical cancer is selected from the group consisting of squamous cell carcinoma and adenocarcinoma.

2. (canceled)

3. (canceled)

4. (canceled)

5. (canceled)

6. (canceled)

7. (canceled)

8. (canceled)

9. The method of claim 1, wherein the heavy chain comprises the amino acid sequence of SEQ ID NO: 9 or SEQ ID NO: 10 and wherein the light chain comprises the amino acid sequence of SEQ ID NO: 11.

10. (canceled)

11. The method of claim 1, wherein the cytotoxic molecule comprises a tubulin inhibitor or DNA damaging agent.

12. The method of claim 11, wherein the cytotoxic molecule comprises a tubulin inhibitor, wherein the tubulin inhibitor comprises a dolastatin or derivative thereof, an auristatin or derivative thereof, or a maytansinoid or derivative thereof.

13. The method of claim 12, wherein the tubulin inhibitor comprises monomethyl auristatin E (MMAE), monomethyl auristatin F (MMAF), or auristatin F (AF).

14. (canceled)

15. (canceled)

16. (canceled)

17. The method of claim 1, wherein the antibody-drug conjugate is represented by formula Ab-(L-U)n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody.

18. (canceled)

19. (canceled)

20. The method of claim 1, wherein the antibody-drug conjugate is disitamab vedotin.

21. (canceled)

22. The method of claim 1, wherein the cervical cancer has been previously treated.

23. The method of claim 22, wherein the cervical cancer has been previously treated with at least first-line platinum-based standard therapy.

24. The method of claim 22, wherein the cervical cancer has been previously treated with radiotherapy.

25. (canceled)

26. (canceled)

27. (canceled)

28. (canceled)

29. (canceled)

30. (canceled)

31. (canceled)

32. (canceled)

33. The method of claim 1, wherein the cancer has a primary FIGO stage of IIB or above.

34. The method of claim 1, wherein the antibody-drug conjugate is administered to the individual at a dose of 2.0 mg/kg.

35. The method of claim 34, wherein the dose is measured using the Bovine serum albumin (BSA)-based Extinction Coefficient (EC) method.

36. (canceled)

37. The method of claim 1, wherein the antibody-drug conjugate is administered to the individuals every 2 weeks or every 14 days.

38. (canceled)

39. (canceled)

40. (canceled)

41. The method of claim 1, wherein administration of the antibody-drug conjugate results in a complete response (CR) or partial response (PR) in the individual.

42. The method of claim 1, wherein the time to onset of effect is less than two months.

43. (canceled)

44. (canceled)

45. The method of claim 1, wherein the duration of progression free survival is longer than four months.

46. (canceled)

47. (canceled)

48. (canceled)

49. A pharmaceutical composition for use in treating a previously treated cervical cancer in an individual, wherein the use comprises administering an effective amount of the anti-HER2 antibody-drug conjugate according to the method of claim 1.

50. A method for treating or preventing progression of cervical cancer in an individual, comprising administering to the individual an effective amount of an anti-human epidermal growth factor receptor 2 (HER2) antibody-drug conjugate that comprises an anti-HER2 antibody and a cytotoxic molecule, and an effective amount of an anti-PD-1 antibody;
wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and
wherein the anti-PD-1 antibody comprises a heavy chain comprising a heavy chain variable (VH) domain and a light chain comprising a light chain variable (VL) domain, wherein the VH domain comprises a CDR-H1 comprising the amino acid sequence STTYYWV (SEQ ID NO: 17), a CDR-H2 comprising the amino acid sequence SISYSGSTYYNPSLKS (SEQ ID NO: 18), and a CDR-H3 comprising the amino acid sequence HLGYNGRYLPFDY (SEQ ID NO: 19) and the VL domain comprises a CDR-L1 comprising the amino acid sequence TGTSSDVGFYNYVS (SEQ ID NO: 20), a CDR-L2 comprising the amino acid sequence DVSNRPS (SEQ ID NO: 21), and a CDR-L3 comprising the amino acid sequence SSYTSISTWV (SEQ ID NO: 22).

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. A kit comprising an anti-human epidermal growth factor receptor 2 (HER2) antibody-drug conjugate and an anti-PD-1 antibody,
wherein the anti-HER2 antibody-drug conjugate comprises an anti-HER2 antibody and a cytotoxic molecule, wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO: 3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO: 5), and a CDR-L3 comprising the amino acid sequence HQFATYT (SEQ ID NO: 6); and
wherein the anti-PD-1 antibody comprises a heavy chain comprising a heavy chain variable (VH) domain and a light chain comprising a light chain variable (VL) domain, wherein the VH domain comprises a CDR-H1 comprising the amino acid sequence STTYYWV (SEQ ID NO: 17), a CDR-H2 comprising the amino acid sequence SISYSGSTYYNPSLKS (SEQ ID NO: 18), and a CDR-H3 comprising the amino acid sequence HLGYNGRYLPFDY (SEQ ID NO: 19) and the VL domain comprises a CDR-L1 comprising the amino acid sequence TGTSSDVGFYNYVS (SEQ ID NO: 20), a CDR-L2 comprising the amino acid sequence DVSNRPS (SEQ ID NO: 21), and a CDR-L3 comprising the amino acid sequence SSYTSISTWV (SEQ ID NO: 22).

112. (canceled)

113. (canceled)


SEQUENCE LISTING (22 sequences)

SEQ ID NO: 1 - protein, 5 aa, synthetic construct
DYYIH

SEQ ID NO: 2 - protein, 17 aa, synthetic construct
RVNPDHGDSYYNQKFKD

SEQ ID NO: 3 - protein, 6 aa, synthetic construct
NYLFDH

SEQ ID NO: 4 - protein, 11 aa, synthetic construct
KASQDVGTAVA

SEQ ID NO: 5 - protein, 7 aa, synthetic construct
WASIRHT

SEQ ID NO: 6 - protein, 7 aa, synthetic construct
HQFATYT

SEQ ID NO: 7 - protein, 115 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 8 - protein, 105 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 9 - protein, 444 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 445 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 212 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 9 aa, synthetic construct
ARNYLFDHW

SEQ ID NO: 13 - protein, 450 aa, synthetic construct
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 123 aa, synthetic construct
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSS

SEQ ID NO: 15 - protein, 216 aa, synthetic construct
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 113 aa, synthetic construct
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVLGQP

SEQ ID NO: 17 - protein, 7 aa, synthetic construct
STTYYWV

SEQ ID NO: 18 - protein, 16 aa, synthetic construct
SISYSGSTYYNPSLKS

SEQ ID NO: 19 - protein, 13 aa, synthetic construct
HLGYNGRYLPFDY

SEQ ID NO: 20 - protein, 14 aa, synthetic construct
TGTSSDVGFYNYVS

SEQ ID NO: 21 - protein, 7 aa, synthetic construct
DVSNRPS

SEQ ID NO: 22 - protein, 10 aa, synthetic construct
SSYTSISTWV